(19) United States
(12) Patent Application Publication
(10) Pub. No.: US 2012/0114755 A1
(43) Pub. Date: May 10, 2012

(54) METHODS AND MATERIALS FOR TISSUE REPAIR

(75) Inventors: Peter C. Amadio, Rochester, MN (US); Chunfeng Zhao, Rochester, MN (US); Steven L. Moran, Rochester, MN (US); Yu-long Sun, Rochester, MN (US); Kai-Nan An, Rochester, MN (US)

(73) Assignee: MAYO FOUNDATION FOR MEDICAL EDUCATION AND RESEARCH, Rochester, MN (US)

(21) Appl. No.: 13/379,299

(22) PCT Filed: Jun. 22, 2010
(86) PCT No.: PCT/US10/39418

§ 371 Date: Dec. 19, 2011

Related U.S. Application Data
(60) Provisional application No. 61/219,621, filed on Jun. 23, 2009.
(60) Provisional application No. 61/219,144, filed on Jun. 22, 2009.

(51) Int. Cl. A61K 9/00; A61P 17/02; A61P 41/00; A61K 35/12

(52) U.S. Cl. 424/486; 424/484; 424/93.7

(57) ABSTRACT

This document relates to methods and materials for treating tendon injury. Specifically, methods and materials for preventing adhesion formation and promoting tissue healing following tendon injury and surgical repair are provided.

### CROSS-REFERENCE TO RELATED APPLICATIONS

This application claims priority to U.S. Provisional Application No. 61/219,621, filed on Jun. 23, 2009, and U.S. Provisional Application No. 61/219,144, filed on Jun. 22, 2009, which are incorporated by reference herein in their entirety.

### STATEMENT AS TO FEDERALLY SPONSORED RESEARCH

This invention was made with government support under grant AR044391 awarded by the National Institute of Arthritis and Musculoskeletal and Skin Disease. The government has certain rights in this invention.

### BACKGROUND

1. Technical Field

This document relates to methods and materials for tissue repair. Specifically, this document provides methods and materials for preventing adhesion formation and promoting tissue healing following surgical tissue repair.

2. Background Information

One of the most common complications following surgical tissue repair is adhesion formation. Adhesions are especially common following abdominal and pelvic surgeries. Adhesions develop when the body's repair mechanisms respond to any tissue disturbance, such as surgery, infection, trauma, or radiation, by connecting, with scar tissue, structures which are normally separated. Although adhesions can occur anywhere, the most common locations are within the stomach, pelvis, and at the site of tendon or ligament damage. Post-operative adhesions can limit active range of motion or impair organ function. Additional surgeries may be required to remove or divide the adhesions, and thereby to restore functionality and range of motion, particularly in the case of tendon and ligament injuries.

The typical tendon injury requires three to four months of rehabilitation, during which time the affected joint is unavailable for work use. Failure rates or residual impairment remain disturbingly high, in the 20-30 percent range in most cases, despite advances in the field. Current methods to prevent adhesions often also impair healing, and have thus found only limited clinical use. Similarly, methods that augment healing may result in increased adhesions. To date, a product which combines both adhesion prevention and augmentation of healing, thus overcoming the limitations of either method alone, has not been conceived.

### SUMMARY

This document provides methods and materials that can be used to repair damaged tissue. For example, the methods and materials provided herein can be used to promote the healing of damaged tendon tissue. As described herein, this document provides methods and materials for generating a composite tissue matrix seeded with stem cells and augmented with structural proteins and, in some cases, an anti-adhesive coating. This document also provides methods and materials for using such a composition for repairing damaged tissue by coating said tissue matrix and/or adjacent tissue (e.g., adjacent undamaged tendon tissue) with an anti-adhesive. As described herein, this document provides, for example, methods and materials by which clinicians and other professionals can contact a stem cell-seeded tissue matrix and an anti-adhesive substance to a tissue at the site of surgical repair in order to reduce surface friction and reduce tissue adhesions while promoting wound healing following surgical repair. Such treatment methods can have substantial value for clinical use.

In general, one aspect of this document features a composition comprising, or consisting essentially of, a tissue matrix and an anti-adhesive coating. The tissue matrix can comprise stem cells and one or more structural polypeptides or one or more biocompatible polymers. The tissue matrix can have an anti-adhesive coating present on at least one surface of the tissue matrix. The coating present on at least one surface of the tissue matrix can not contact a wound or sutured tissue after implantation. The wound or sutured tissue can be tendon, ligament, abdominal, uterine, or muscle tissue. The one or more structural proteins can be selected from the group consisting of a collagen, a proteoglycan, and a cytokine, and any combination thereof. The one or more structural polypeptides can be selected from the group consisting of collagen, aggregan, versican, decorin, biglycan, fibromodulin, lumican, IL-1, IL-6, and TNF-α, and any combination thereof. The tissue matrix can be an acellular tissue scaffold. The tissue matrix can be a collagen matrix. The collagen matrix can be a matrix of bioengineered collagen fibers. The anti-adhesive coating can be selected from the group consisting of lubricin, hyaluronic acid, phospholipids, and any combination thereof. The lubricin can be native human lubricin. The lubricin can be native canine lubricin. The lubricin can be recombinant lubricin. The stem cells can be autologous stem cells. The stem cells can be derived from muscle, skin, bone marrow, synovium, or adipose tissue. The stem cells can be mesenchymal stem cells. The mesenchymal stem cells can be bone marrow stromal cells. The composition can be an implantable patch. The composition can further comprise a growth factor selected from the group consisting of transforming growth factor (TGF-β1), platelet derived growth factor (PDGF), basic fibroblast growth factor (b-FGF), insulin like growth factor (IGF), epidermal growth factor (EGF), growth differentiation factor 5 (GDF-5), growth differentiation factor 6 (GDF-6), growth differentiation factor 7 (GDF-7), and vascular endothelial growth factor (VEGF), and any combination thereof. The composition can further comprise a neuropeptide. The neuropeptide can be substance P. The composition can further comprise platelet-rich plasma.

In another aspect, this document features a method for providing an implantable patch to a mammal, e.g., to repair diseased or damaged tissue. The method comprises, or consists essentially of, implanting a composition, e.g., a tissue matrix, as described above. In some embodiments, an anti-adhesive coating can be present on at least one surface of the tissue matrix that does not contact the diseased or damaged tissue after implantation. In some embodiments, an anti-adhesive is applied to the tissue matrix and/or tissue adjacent to the diseased or damaged discuss after implanting of the tissue matrix. The anti-adhesive so applied does not contact diseased or damaged tissue (e.g., the wound or sutured tissue), but may contact undamaged or undiseased tissue adjacent to the tissue matrix. The implantable patch can repair tissue damage. The implantable patch can prevent tissue adhesion. The implantable patch can prevent leakage of the anti-adhesive coating into the wound or the sutured tissue. One or more structural polypeptides included in the patch can be selected from the group consisting of collagen, aggregan, versican, decorin, biglycan, fibromodulin, lumican, IL-1, IL-6, and TNF-α, and any combination thereof. The diseased or damaged tissue can be tendon, ligament, abdominal, uterine, or muscle tissue. The anti-adhesive coating can be selected from the group consisting of lubricin, hyaluronic acid, phospholipids, and any combination thereof. The lubricin can be native human lubricin. The lubricin can be native canine lubricin. The lubricin can be recombinant lubricin. The stem cells can be autologous stem cells. The stem cells can be derived from muscle, skin, bone marrow, synovium, or adipose tissue. The stem cells can be mesenchymal stem cells. The mesenchymal stem cells can be bone marrow stromal cells. The method can further comprise a growth factor selected from the group consisting of transforming growth factor (TGF-β1), platelet derived growth factor (PDGF), basic fibroblast growth factor (b-FGF), insulin like growth factor (IGF), epidermal growth factor (EGF), growth differentiation factor 5 (GDF-5), growth differentiation factor 6 (GDF-6), growth differentiation factor 7 (GDF-7), and vascular endothelial growth factor (VEGF), and any combination thereof. The method can further comprise a neuropeptide. The neuropeptide can be substance P. The method can further comprise platelet-rich plasma.

In another aspect, this document features a method for treating a wound or sutured tissue comprising, or consisting essentially of, contacting a tissue matrix to a wound or sutured tissue. The tissue matrix can comprise one or more stem cells and one or more structural polypeptides or one or more biocompatible polymers. The method can comprise coating at least a portion of the tissue matrix and/or adjacent non-wound or non-sutured tissue with an anti-adhesive. The coating of anti-adhesive can not contact a wound or sutured tissue. The tissue matrix can prevent leakage of the anti-adhesive into the wound or sutured tissue. The wound or sutured tissue can be tendon, ligament, abdominal, uterine, or muscle tissue. The one or more structural proteins can be selected from the group consisting of a collagen, a proteoglycan, and a cytokine, and any combination thereof. The one or more structural polypeptides can be selected from the group consisting of collagen, aggregan, versican, decorin, biglycan, fibromodulin, lumican, IL-1, IL-6, and TNF-α, and any combination thereof. The tissue matrix can be an acellular tissue scaffold. The tissue matrix can be a collagen matrix. The collagen matrix can be a matrix of bioengineered collagen fibers. The wound or sutured tissue can be tendon, ligament, abdominal, uterine, or muscle tissue. The anti-adhesive coating can be selected from the group consisting of lubricin, hyaluronic acid, phospholipids, platelet-rich plasma, and any combination thereof. The lubricin can be native human lubricin. The lubricin can be native canine lubricin. The lubricin can be recombinant lubricin. The stem cells can be autologous stem cells. The stem cells can be derived from muscle, skin, bone marrow, synovium, or adipose tissue. The stem cells can be mesenchymal stem cells. The mesenchymal stem cells can be bone marrow stromal cells. The method can further comprise a growth factor selected from the group consisting of transforming growth factor (TGF-β1), platelet derived growth factor (PDGF), basic fibroblast growth factor (b-FGF), insulin like growth factor (IGF), epidermal growth factor (EGF), growth differentiation factor 5 (GDF-5), growth differentiation factor 6 (GDF-6), growth differentiation factor 7 (GDF-7), and vascular endothelial growth factor (VEGF), and any combination thereof. The method can further comprise a neuropeptide. The neuropeptide can be substance P. The method can further comprise platelet-rich plasma.

In a further aspect, this document features a method for treating a wound or sutured tissue comprising, or consisting essentially of, contacting a composition to a wound or sutured tissue. The composition can comprise a tissue matrix comprising one or more stem cells, one or more structural polypeptides or one or more biocompatible polymers, and an anti-adhesive coating. The wound or sutured tissue can be treated. The anti-adhesive coating is present on at least one surface of said tissue matrix. The anti-adhesive coating does not contact a wound or sutured tendon tissue. The method can further comprise further coating the composition and/or adjacent non-wound and non-sutured tissue with the anti-adhesive coating following contacting of the composition to the wound or sutured tissue. The wound or sutured tissue can be tendon, ligament, abdominal, uterine, or muscle tissue. The one or more structural proteins can be selected from the group consisting of a collagen, a proteoglycan, and a cytokine, and any combination thereof. The one or more structural polypeptides can be selected from the group consisting of collagen, aggregan, versican, decorin, biglycan, fibromodulin, lumican, IL-1, IL-6, and TNF-α, and any combination thereof. The tissue matrix can be an acellular tissue scaffold. The tissue matrix can be a collagen matrix. The collagen matrix can be a matrix of bioengineered collagen fibers. The wound or sutured tissue can be tendon, ligament, abdominal, uterine, or muscle tissue. The anti-adhesive coating can be selected from the group consisting of lubricin, hyaluronic acid, phospholipids, platelet-rich plasma, and any combination thereof. The lubricin can be native human lubricin. The lubricin can be native canine lubricin. The lubricin can be recombinant lubricin. The stem cells can be autologous stem cells. The stem cells can be derived from muscle, skin, bone marrow, synovium, or adipose tissue. The stem cells can be mesenchymal stem cells. The mesenchymal stem cells can be bone marrow stromal cells. The method can further comprise a growth factor selected from the group consisting of transforming growth factor (TGF-β1), platelet derived growth factor (PDGF), basic fibroblast growth factor (b-FGF), insulin like growth factor (IGF), epidermal growth factor (EGF), growth differentiation factor 5 (GDF-5), growth differentiation factor 6 (GDF-6), growth differentiation factor 7 (GDF-7), and vascular endothelial growth factor (VEGF), and any combination thereof. The method can further comprise a neuropeptide. The neuropeptide can be substance P. The method can further comprise platelet-rich plasma.

In another aspect, this document features a method of promoting healing of a tissue injury in a mammal. The method comprises, or consists essentially of, contacting a composition to a tissue injury following surgical repair. The composition can comprise a tissue matrix comprising one or more stem cells and one or more structural proteins or one or more biocompatible polymers and optionally an anti-adhesive coating. The anti-adhesive coating can be present on at least one surface of the tissue matrix that does not contact the tissue injury. The method can further include coating the tissue matrix and/or adjacent tissue to the tissue injury with an anti-adhesive. The contacting can promote healing of the tissue injury. The healing of the tissue injury does not comprise or reduces adhesion formation.

In another aspect, this document features a method of treating a tissue injury in a mammal, comprising contacting a tissue matrix to the tissue injury following surgical repair. The tissue matrix can comprise one or more stem cells, one or more structural proteins or one or more biocompatible polymers, and optionally an anti-adhesive coating. The anti-adhesive coating can be present on at least one surface of the tissue matrix that does not contact the tissue injury. The method can optionally include further coating the tissue matrix and/or adjacent tissue to the tissue injury with an anti-adhesive. The contacting can treat the tissue injury.

In a further aspect, this document features an article of manufacture. The article of manufacture comprises, or consists essentially of, packaging material, a composition as described herein, an anti-adhesive, and written instructions for using the composition and the anti-adhesive for tissue repair.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph representing gliding resistance after canine peroneus longus tendon surface modification with one of the following solutions: saline solution, lubricin, carbodiimide derivatized gelatin (cd-G), carbodiimide derivatized gelatin with hyaluronic acid (cd-HAG), or carbodiimide derivatized gelatin to which lubricin had been added in a second step (cd-G+lubricin).

FIG. 2 is a graph representing human peroneus longus tendon gliding resistance before and after surface treatment with one of the following solutions: saline solution (control), cd-G, cd-HAG, or cd-G to which lubricin had been added in a second step (cd-G+lubricin).

FIG. 3 is a graph representing normalized gliding resistance after flexor tendon repair with one of the following solutions: saline solution, cd-HA-gelatin (cd-HAG), cd-gelatin+lubricin (cd-G+lubricin), and cd-HA-gelatin+lubricin (cd-HAG+lubricin).

FIG. 4 contains photographs depicting tendon surface examined by SEM. Note rough surface in saline control group (A) and smooth surface in cd-HAG+lubricin group (B).

FIG. 5 is a bar graph representing a comparison of normalized work of flexion (nWOF) in three groups at three time points.

FIG. 6 is a graph representing the contraction rate for four collagen gel concentrations (0.5, 1.0, 1.5, 2.0 mg/mL) seeded with BMSC at cell density of 1.0×10⁶ cells/mL. Gel contraction was evaluated after 0.5, 1, 2, 3, 4, 5, 6, and 7 days in culture.

FIG. 7 is a graph representing the effect of gel concentration on mechanical properties of the contracted gel ring.

FIG. 8 contains photographs of BMSC distribution in a collagen gel patch after 12, 24, and 48 hours in culture.

FIG. 9 is a graph representing the effect of cell density on the rate of gel contraction. Cell densities of 0.1×10⁶ cells/mL, 0.25×10⁶ cells/mL, 0.5×10⁶ cells/mL, and 1.0×10⁶ cells/mL were assayed over 13 days.

FIG. 10 is a graph representing the effect of cell density on mechanical properties of a contracted gel ring. Cell densities of 0.1×10⁶ cells/mL, 0.25×10⁶ cells/mL, 0.5×10⁶ cells/mL, and 1.0×10⁶ cells/mL were assayed for mechanical properties.

FIG. 11 is a photograph depicting tissue culture of repaired tendons+BMSC-seeded collagen-gel patch. Isolated canine BMSC, at an initial concentration of 1×10⁶ cells, were seeded into 0.5 mg/mL of collagen. Repaired canine flexor digitorum profundus (FDP) tendons were mounted on a square frame with four pairs of clamps to maintain tendon in a straight position during tissue culture.

FIG. 12 is a graph representing ultimate failure strength of repaired tendons+BMSC-seeded collagen-gel patch.

FIG. 13 is a photograph depicting a BMSC-seeded gel patch. BMSC were labeled with PKH26 red fluorescent cell linker before seeding to the gel patch. Viable cells were detected between tendon ends by red fluorescence following two weeks in tissue culture.

FIG. 14 is a photograph depicting tissue culture of the repaired tendon with gel patch.

FIG. 15 is a photograph depicting a tendon mounted on the micro-tester. Before the tendon was distracted, the sutures were cut to assess the strength of the healing tissue.

FIG. 16 is a bar graph depicting a MTT assay. Each graph presents mean+SD from a representative experiment performed in triplicate. *, P<0.05.

FIG. 17 is a series of graphs demonstrating the results of quantitative RT-PCR. Each graph shows the expression of tenomodulin (A), collagen type I (B), collagen type III (C). Results are presented as mean+SD of n=5. *, P<0.05.

FIG. 18 is a series of graphs demonstrating ultimate force (A) and stiffness (B). Each graph represents mean+SD of n=8. *, P<0.05; **, P<0.01. 76×96 mm (300×300 DPI).

FIG. 19 is a series of photographs depicting histology of the repair tissue at 4 weeks. Each panel shows repaired tendon without gel patch (A), repaired tendon with cell-seeded gel patch (B), repaired tendon with GDF5 added gel patch without cells (C), repaired tendon with GDF5 treated cell-seeded gel patch (D). 101×99 mm (300×300 DPI).

FIG. 20 is a set of graphs demonstrating (A) maximum strength of the healing tendon (mean±SD. *=p<0.01, **=p<0.02), and (B) stiffness of the healing tendon (mean±SD. *=p<0.01, **=p<0.02).

FIG. 21 is a series of photographs showing labeled BMSC with PKH26 cell linker as observed under confocal microscopy with red fluorescence. (A) BMSC-seeded patch at 2 weeks, (B) BMSC-seeded PRP patch at 2 weeks, (C) BMSC-seeded patch at 4 weeks, (D) BMSC-seeded PRP patch at 4 weeks.

FIG. 22 is a series of photographs depicting the healing tendons stained with hematoxylin and eosin at 2 weeks. FIG. 22A: (a) No patch group, (b) PRP patch group, (c) BMSC-seeded patch group, and (d) BMSC-seeded PRP patch group. Scale=0.5 mm. FIG. 22B: the healing tendons stained with hematoxylin and eosin at 4 weeks. (a) No patch group, (b) PRP patch group, (c) BMSC-seeded patch group, and (d) BMSC-seeded PRP patch group. Scale=0.5 mm.

### DETAILED DESCRIPTION

This document relates to methods and materials involved in tissue repair. As described herein, this document also provides methods and materials for generating a tissue matrix seeded with stem cells and augmented with structural proteins and, in some cases, an anti-adhesive coating either before or after implantation. The methods and materials provided herein can be used to reduce surface friction and reduce tendon and other tissue adhesions while promoting wound healing following surgical repair.

### Composition

This document provides methods and materials for a preparing a composition comprising a tissue matrix. Any appropriate materials can be used to prepare such a composition. In some cases, biological materials such as, for example, Type I collagen fibers can be used as a tissue matrix. Type I collagen can be isolated and purified from Type I collagen-rich tissues such as skin, tendon, ligament, and bone of humans and animals as previously described. See, e.g., Miller et al., Methods Enzymol. 82:33-64 (1982); U.S. Pat. No. 6,090,996. Other biopolymeric materials, which can be either natural or synthetic, can be used as a tissue matrix. Biopolymeric materials can include, without limitation, other types of collagen (e.g., type II to type XXI), elastin, fibrin, peptides, polysaccharide (e.g., chitosan, alginic acid, cellulose, and glycosaminoglycan), a synthetic analog of a biopolymer by genetic engineering techniques, a biocompatible polymer, or a combination thereof. Biocompatible polymers can include natural or synthetic biodegradable polymers (e.g., poly(ethylene glycol fumarate)). Vitrogen bovine dermal collagen (Cohesion Technologies, Palo Alto, Calif.) can be used. In some cases, genetically engineered collagens such as those marketed by Fibrogen (South San Francisco, Calif.) or from cell culture techniques such as those described by Advanced Tissue Sciences (La Jolla, Calif.) can be used. In some cases, a tissue matrix can be a composite of native or bioengineered collagen fibers suspended in a gelatin solution. Any appropriate collagen-gel concentration (e.g., from 0.5 to 2.0 mg/mL) can be used.

In some cases, a tissue matrix can be an acellular tissue scaffold developed from any appropriate decellularized tissue. For example, tissue such as tendon or ligament tissue can be decellularized by appropriate method to remove native cells from the tissue while maintaining morphological integrity of the tissue portions and preserving extracellular matrix (ECM) proteins. Decellularization methods can include subjecting tendon and ligament tissue to repeated freeze-thaw cycles using liquid nitrogen or chemical methods such as sodium dodecyl sulfate (SDS). The tissue can also be treated with a nuclease solution (e.g., ribonuclease, deoxyribonuclease) and washed in sterile phosphate buffered saline with mild agitation.

In some cases, a tissue matrix can be seeded with other cells. Any appropriate cell type, such as naïve or undifferentiated cell types, can be used to seed the tissue matrix. Stem cells appropriate for the methods and materials provided herein can include bone marrow mesenchymal stromal cells (BMSC). Stem cells derived from other tissues also can be used. For example, stem cells derived from skin, bone, muscle, bone marrow, synovium, or adipose tissue can be used to develop stem cell-seeded tissue matrices. Any appropriate method for isolating and collecting cells for seeding can be used. For example, bone marrow stromal cells generally can be harvested from bone marrow. Isolated cells can be rinsed in a buffered solution (e.g., phosphate buffered saline) and resuspended in a cell culture medium. Standard cell culture methods can be used to culture and expand the population of cells. Once obtained, the cells can be contacted with a tissue matrix to seed the matrix. For example, a tissue matrix can be seeded with cells in vitro at any appropriate cell density. For example, cell densities from 0.2×10⁶ to about 1×10⁷ cells/matrix can be used. In some cases, a collagen solution can be combined with cultured cells and the cell density in the tissue matrix can be adjusted to an initial cell density of about 1.0×10⁶ cells/mL. The seeded tissue matrix can be incubated for a period of time (e.g., from several hours to about 14 days) post-seeding to improve fixation and penetration of the cells in the tissue matrix. Histology and cell staining can be performed to assay for seeded cell propagation. Any appropriate method can be performed to assay for seeded cell differentiation. For example, quantitative real-time reverse transcription-polymerase chain reaction (RT-PCR) can be performed to detect and measure expression levels of markers of tenocyte differentiation (e.g., tenomodulin), gelatinase (e.g., MMP2), and collagenase (e.g., MMP13).

In some cases, a tissue matrix can be augmented with one or more structural polypeptides including, for example, collagen (e.g., Type I, Type II, Type III, and Type IV collagen), and proteoglycans (e.g., aggregan, versican, decorin, biglycan, fibromodulin, or lumican). In some cases, a tissue matrix can be impregnated with one or more growth factors or neuropeptides to stimulate differentiation of the seeded cells. For example, a tissue matrix can be impregnated with the growth factor TGF-β1. Other growth factors appropriate for the methods and materials provided herein can include, for example: platelet derived growth factor (PDGF), basic fibroblast growth factor (b-FGF), insulin like growth factor (IGF), epidermal growth factor (EGF), growth differentiation factor-5 (GDF-5), growth differentiation factor 6 (GDF-6), growth differentiation factor (GDF-7), and vascular endothelial growth factor (VEGF). Neuropeptides appropriate for the methods and materials provided herein can include, for example, substance P(SP) and neuropeptide Y. In some cases, a tissue matrix can be impregnated with platelet-rich plasma to aid in, for example, the differentiation of seeded cells.

Polypeptides for the methods and materials provided herein can be obtained by any appropriate method. By way of example and without limitation, a structural polypeptide can be obtained by expression of a recombinant nucleic acid encoding the polypeptide or by chemical synthesis (e.g., by solid-phase synthesis or other methods well known in the art, including synthesis with an ABI peptide synthesizer; Applied Biosystems, Foster City, Calif.). In some cases, expression vectors that encode the polypeptide of interest can be used to produce a polypeptide. For example, standard recombinant technology using expression vectors encoding a polypeptide can be used. Expression systems that can be used for small or large-scale production of the polypeptides provided herein include, without limitation, microorganisms such as bacteria transformed with recombinant bacteriophage DNA, plasmid DNA, or cosmid DNA expression vectors containing the nucleic acid molecules of the polypeptide of interest. The resulting polypeptides can be purified according to any appropriate protein purification method. In some cases, commercially-available recombinant polypeptides (e.g., recombinant GDF-5 from R&D systems, Minneapolis, Minn.) can be used to augment a tissue matrix.

Structural polypeptides, growth factors, platelet-rich plasma, and/or neuropeptides can be added to biopolymeric materials at any step in the tissue matrix-making process. In some cases, polypeptides can be added when preparing a composite of native or bioengineered collagen fibers suspended in a gelatin solution. In some cases, polypeptides can be added to a prepared tissue matrix comprising a composite of native or bioengineered collagen fibers suspended in a gelatin solution. Structural polypeptides can be added to a prepared tissue matrix just prior to contacting the tissue matrix to tissue for in vivo tissue repair. Structural polypeptides can be added to a cell-seeded tissue matrix at any appropriate concentration. For example, the concentration of one or more structural polypeptides can vary from 50 to 500 ng/mL.

This document also provides methods and materials for a tissue matrix comprising an anti-adhesive coating. Any appropriate anti-adhesive can be used. For example, an anti-adhesive coating can be lubricin, hyaluronic acid, or phospholipids. Lubricin is a proteoglycan found in synovial fluid and in the superficial zone of articular cartilage. Lubricin has both lubricating and anti-cellular adhesion properties. Hyaluronic acid (HA), a polysaccharide, is found in all vertebrate tissues and body fluids. Various physiological functions have been assigned to HA, including lubrication, water homeostasis, filtering effects, and regulation of plasma protein distribution. See Fraser et al., J. Intern. Med. 242(1):27-33 (1997). Like lubricin, phospholipids have lubricating and anti-cellular adhesion properties.

In some cases, an anti-adhesive coating can be an anti-adhesive combined with a water-soluble proteinacious polymer (e.g., gelatin). For example, an anti-adhesive coating can be a gelatin polymer gel containing lubricin, HA, and/or phospholipids. In some cases, a water-soluble carbodiimide such as 1-ethy 1-3-(3-dimethylaminopropyl)carbodiimide hydrochloride (EDC) can be used to modify, and thereby increase the half-life of, an anti-adhesive. For example, an anti-adhesive coating can be a gelatin polymer gel containing carbodiimide-derivatized HA, or carbodiimide-derivatized HA supplemented with lubricin. Derivatized HA is commercially available as a cross-linked gel (Hyaloglide® ACP gel, Fidia Advanced Biopolymers, Abano Terme, Italy).

In some cases, the composition can be an implantable patch. For example, the composition can be an implantable gel patch for implanting into the site of tissue repair. In some cases, an anti-adhesive coating is applied to a surface of the implantable patch that does not extend to the damaged or injured tissue prior to implantation. In some cases, an anti-adhesive coating is applied to a surface of the implantable patch that does not extend to the damaged or injured tissue following implantation to the site of tissue repair. When implanted as a gel patch at the site of tissue repair, the coated or uncoated composition is contacted to the damaged or injured tissue and, in some cases, surfaces of the composition that are not in contact with the damaged or injured tissue can be further coated with an anti-adhesive. The anti-adhesive coated surface(s) remain exposed to surrounding tissues. In this manner, the implantable patch can serve as a barrier to prevent leakage of an anti-adhesive coating into the site.

This document also provides articles of manufacture that can include any of the compositions described herein. For example, any of the compositions described herein can be combined with packaging material to generate articles of manufacture or kits. Components and methods for producing articles of manufacture are well known. In addition to a tissue matrix composition, an article of manufacture further can include, for example, one or more anti-adhesives, sterile water, pharmaceutical carriers, buffers, and/or other reagents for treating or repairing tissue. In addition, printed instructions describing how the composition contained therein can be used to treat or repair tissue can be included in such articles of manufacture.

The components in an article of manufacture can be packaged in a variety of suitable containers. In some cases, an article of manufacture can include composition as described herein in a pre-packaged form in quantities sufficient for a single administration or for multiple administrations in, for example, sealed pouches, sealed ampoules, capsules, or cartridges. Such containers can be air tight and/or waterproof, and can be labeled for use.

### Methods for Using Composition

This document also provides methods and materials for repairing damaged tissue. Any appropriate tissue can be repaired according to the methods provided herein. For example, the tissue can be any tissue for which tissue adhesion presents a problem following surgical repair. In some cases, tissue can be tendon, ligament, muscle, uterine, or abdominal tissue. For example, tissue can be the muscles and tendons of a rotator cuff, and damaged tissue can be a torn rotator cuff. Tendons that can be repaired or replaced by the methods described herein can include, for example, the supraspinatus tendon, infraspinatus tendon, Achilles tendon, tibialis anterior tendon, peroneus longus tendon, peroneus medius tendon, extensor digitorum longus tendons, extensor hallucis longus tendon, flexor digitorum longus tendon, or patellar tendon. Ligaments that can be repaired or replaced by the methods described herein can include, for example, the ulnar collateral ligament, radial collateral ligament, medical collateral ligament, lateral collateral ligament, anterior cruciate ligament, posterior cruciate ligament, anterior or posterior talofibular ligaments, calcaneofibular ligament, talocalcaneal ligament, or posterior talocalcaneal ligament.

In some cases, a tissue matrix can be contacted to the site of tissue damage. For example, a tissue matrix can be contacted to the lacerated ends of tendons or ligaments. Contacting can occur prior to, during, or following surgical repair (e.g., suturing) of lacerated tissue. In order to prevent tissue adhesion and seepage of an anti-adhesive between lacerated ends of the tissue, surfaces of the tissue matrix that will not contact the repaired wound or damaged tissue can have an anti-adhesive coating applied either prior to or after implantation, or both prior to and following implantation. With the tissue matrix contacting the tissue, an anti-adhesive coating can be applied to the top of the matrix and to the surrounding tissue. The benefits of this method are two-fold: the tissue matrix provides a passive barrier to prevent anti-adhesive leakage into the wound site, but also actively promotes wound healing and prevents the adhesion of the wounded tissue to surrounding soft tissue during wound healing. In some cases, the anti-adhesive can be coated onto a surface of tissue matrix prior to contacting the tissue matrix to damaged tissue. Alternatively or additionally, an anti-adhesive coating can be applied to a surface of a tissue matrix and, in some cases, to tissue surrounding the tissue matrix, after the tissue matrix has been contacted to damaged tissue.

Any appropriate method(s) can be performed to assay for tissue repair. For example, methods can be performed to assess tissue healing, to assess functionality of repaired tissue, and to assess cellular ingrowth. To determine the extent of tissue healing, histology and cell staining can be performed to detect seeded cell propagation and/or improved histological appearance. In some cases, tissue portions can be collected and treated with a fixative such as, for example, neutral buffered formalin. Such tissue portions can be dehydrated, embedded in paraffin, and sectioned with a microtome for histological analysis. Sections can be stained with hematoxylin and eosin (H&E) and then mounted on glass slides for microscopic evaluation of morphology and cellularity.

In some cases, physiological tests can be performed to assess tissue movement and functionality following treatment according to the methods and materials provided herein. For example, in vitro mechanical assays can be performed to measure the work of flexion (WOF) or flexion angle of repaired tissue. Gross evaluations can be performed to detect adhesion formation at or near the repair site. In vivo assays can include functional evaluation of the organs, symptom assessment, or imaging techniques.

In some cases, RT-PCR techniques can be used to quantify the expression of metabolic and differentiation markers. For example, RT-PCR and real-time RT-PCR can be used to measure the expression of Type I collagen, Type III collagen, fibronectin, TGF-β1, or tenomodulin. In some cases, gene expression of scleraxis, a genetic marker for connective tissue such as tendon and ligament, can be measured. Any appropriate RT-PCR protocol can be used. Briefly, total RNA can be collected by homogenizing a biological sample (e.g., tendon sample), performing a chloroform extraction, and extracting total RNA using a spin column (e.g., RNeasy® Mini spin column (QIAGEN, Valencin, Calif.)) or other nucleic acid-binding substrate.

The invention will be further described in the following examples, which do not limit the scope of the invention described in the claims.

### EXAMPLES

### Example 1

### Lubricin Surface Modification Improves Extrasynovial Tendon Gliding

Forty peroneus longus tendons, along with the proximal pulley in the ipsilateral hind paw, were harvested from adult mongrel dogs. After the gliding resistance of the normal tendons was measured, the tendons were treated with one of the following solutions: saline solution, lubricin, carbodiimide derivatized gelatin (cd-G), carbodiimide derivatized gelatin with hyaluronic acid (cd-HAG), or carbodiimide derivatized gelatin to which lubricin had been added in a second step (cd-gelatin plus lubricin). Tendon gliding resistance was measured for 1000 cycles of simulated flexion-extension motion of the tendon. Transverse sections of the tendons were examined qualitatively at 100× magnification to estimate surface smoothness after 1000 cycles.

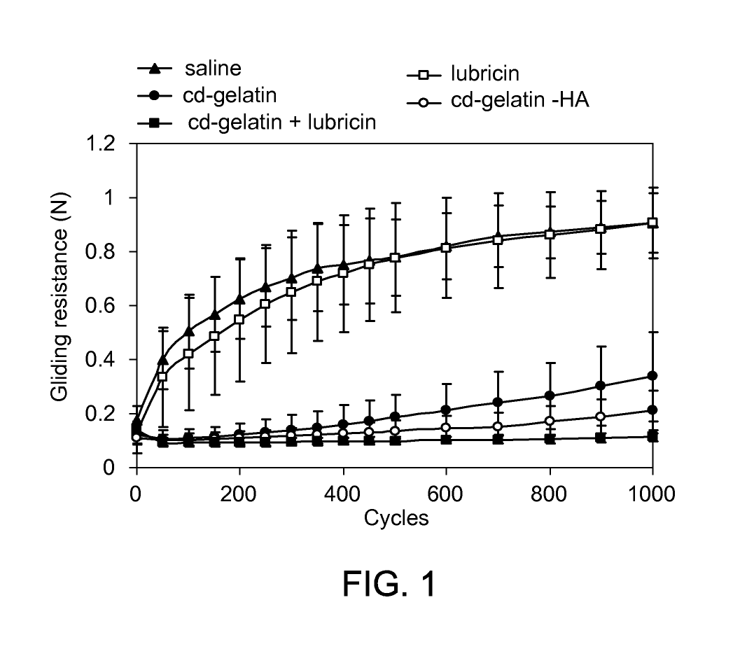

There was no significant difference in the gliding resistance between the tendons treated with saline solution and those treated with lubricin alone, or between the tendons treated with cd-HAG and those treated with cd gelatin plus lubricin; however, the gliding resistance of the tendons treated with cd-gelatin plus lubricin was significantly lower than that of the tendons treated with saline solution, lubricin alone, or cd-gelatin alone (p<0.05) (FIG. 1). After 1000 cycles of tendon motion, the gliding resistance of the tendons treated with cd-gelatin plus lubricin decreased 18.7% compared with the resistance before treatment, whereas the gliding resistance of the saline-solution-treated controls increased>400%.

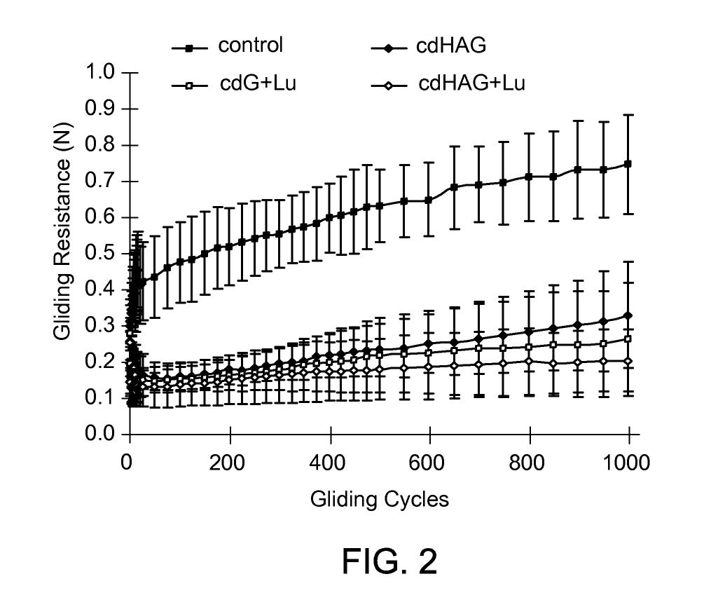

In addition, the tendon surfaces treated with cd-gelatin plus lubricin or with cd-HA-gelatin appeared smooth even after 1000 cycles of tendon motion, whereas the other surfaces appeared roughened. Thus, while the addition of lubricin alone did not affect friction in this tendon gliding model, the results indicate that lubricin may preferentially adhere to a tendon surface pretreated with cd-gelatin and, when so fixed in place, lubricin does have an important effect on tendon lubrication. Comparable results were obtained when the above assays were repeated using an analogous human tendon, the palmaris longus (FIG. 2).

### Example 2

### Lubricin Surface Modification Improves Tendon Gliding after Tendon Repair in a Canine Model

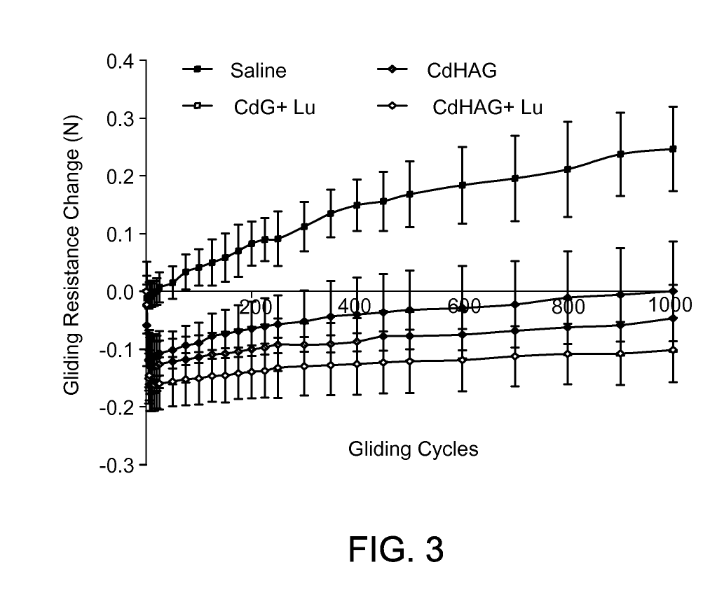

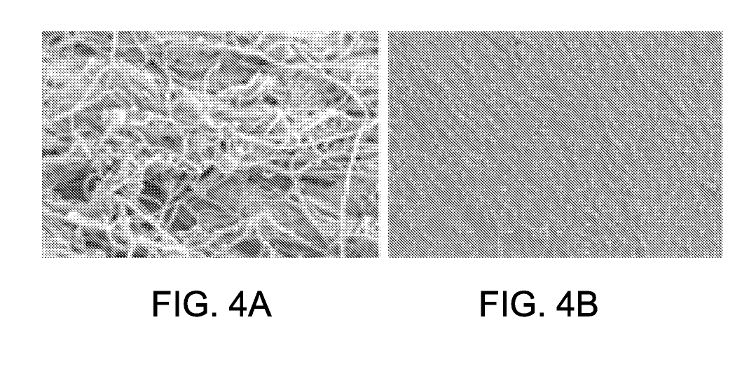

Thirty-two canine FDP tendons from the second, third, fourth, and fifth digits were completely lacerated and repaired with a modified Pennington technique. After the gliding resistance of the repaired tendon was measured, the tendons were treated with one of the following solutions: saline, carbodiimide derivatized-HA-gelatin (cd-HA-gelatin), carbodiimide derivatized-gelatin plus lubricin (cd-gelatin+lubricin), and carbodiimide derivatized gelatin/HA plus lubricin (cd-HA-gelatin+lubricin). After treatment, tendon gliding resistance was measured for 1000 cycles of simulated flexion/extension tendon motion. The surface of the repaired tendon and its proximal pulley was then assessed qualitatively for surface smoothness by scanning electron microscopy (SEM) after 1000 cycles. The increase in average and peak gliding resistance in cd-HA-gelatin, cd-gelatin-lubricin, and cd-HA-gelatin+lubricin tendons was significantly less than that of the saline control tendons after 1000 cycles (p<0.05). The increase in average gliding resistance of cd-HA-gelatin+lubricin treated tendons was also significantly less than that of the cd-HA-gelatin treated tendons (FIG. 3). The surface of the repaired tendons and their proximal pulleys appeared smooth even after 1000 cycles of tendon motion for the cd-HA-gelatin, cd-gelatin+lubricin, and cd-HA-gelatin+lubricin treated tendons, while that of the saline control appeared roughened (FIG. 4). These results suggest that tendon surface modification can improve tendon gliding ability, with a trend suggesting that lubricin fixed on the repaired tendon may provide additional improvement over that provided by HA alone.

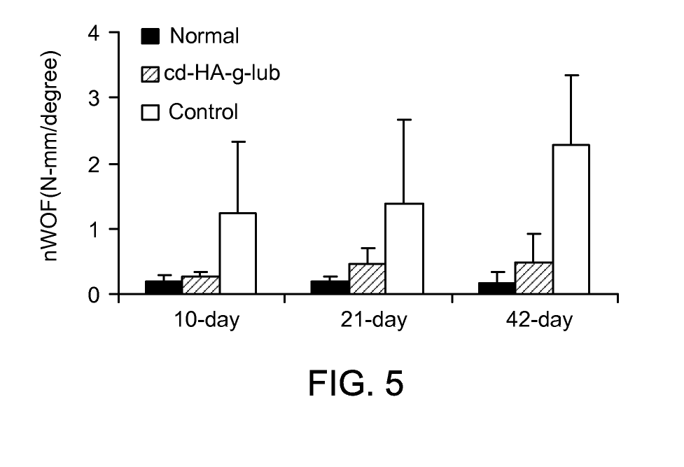

To investigate the effects of physicochemical modification of the tendon gliding surface on tendon healing, operations were performed using a modified Pennington surgical technique. Each repaired tendon was immediately treated with cd-HA-gelatin-lubricin as described above. After 5 days immobilization, a standard therapy of controlled, synergistic movement was instituted, twice daily, 7 days per week. See Zhao et al., J. Bone & Joint Surg., 84:78-84 (2002). The dogs were sacrificed at 10, 21, and 42 days post-operatively. The repaired digit was dissected and digit function was evaluated by measuring the work of flexion normalized by joint motion (nWOF). See Yang et al., J. Biomed. Materials Res. Part B, Applied Biomaterials, 68(1):15-20 (2004). The nWOF of repaired tendons treated with cd-HA-g-lubricin was significantly lower than the control group (repaired tendon without treatment) (p<0.05). See FIG. 5. Importantly, there was no significant difference between the work of flexion of the normal digit and the tendon repair with cd-HA-g-lubricin augmentation group, thus indicating the restoration of near normal gliding resistance following tendon repair in vivo. While the rate of tendon rupture and gap formation was higher in the cd-HAG-lubricin treated group (rupture: 22%, gap 33%) compared with control group (rupture: 3%, gap: 25%), these data suggested that lubricin improved digit function and decreased adhesion formation.

### Example 3

### Bone Marrow Stromal Cell Seeded Gel Matrix and Tendon Repair

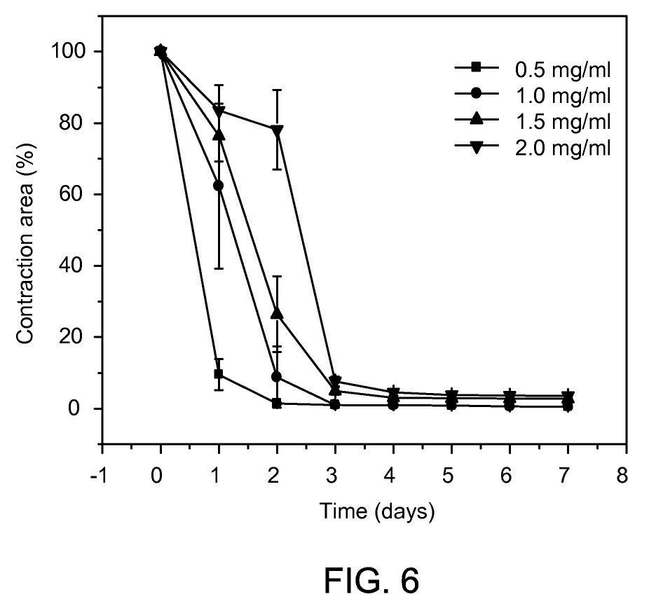

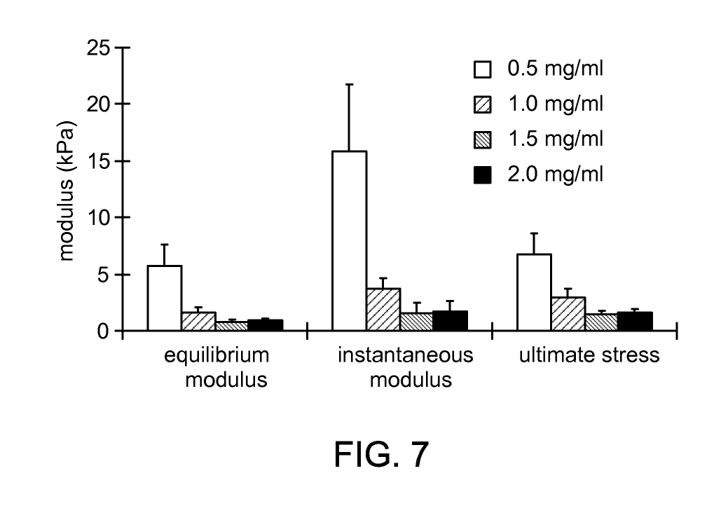

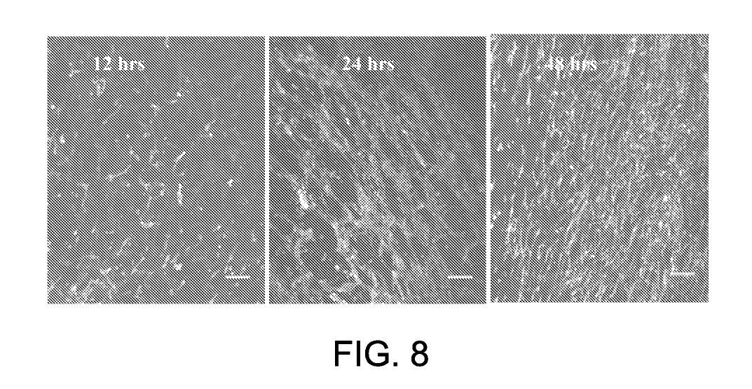

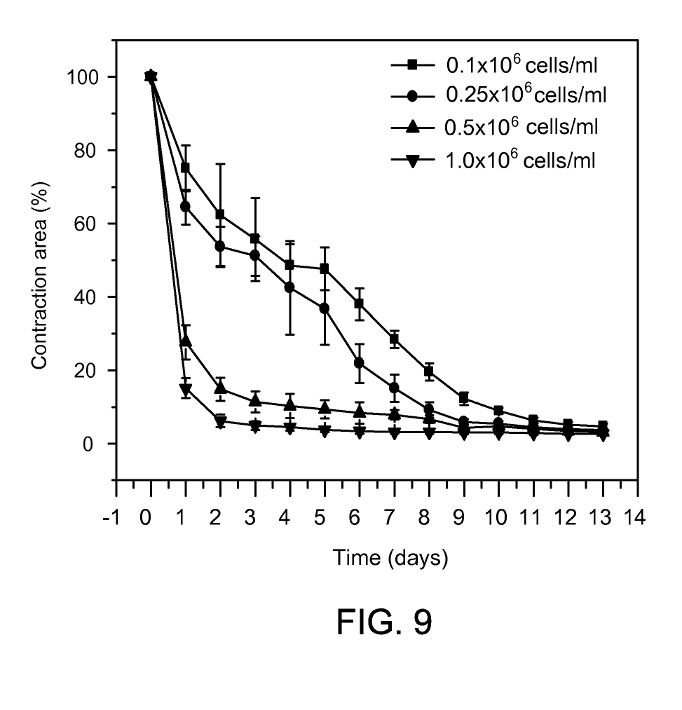

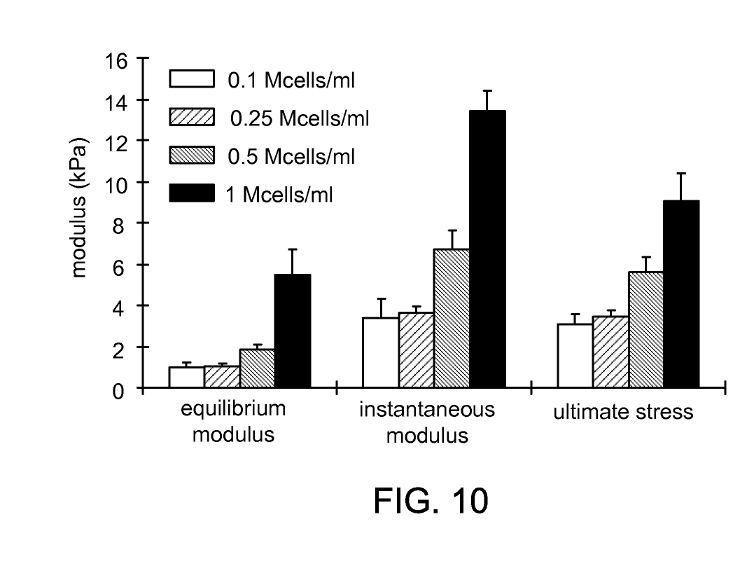

To investigate methods of preserving the anti-adhesive effects of surface modification with lubricin and HA while augmenting the healing potential of the repair with an additional tissue engineering approach, a collagen patch augmented with bone marrow stromal cells (BMSC) was used. To develop the patch, BMSCs passaged up to four times were used. The stem cells were mixed with type I purified bovine dermal collagen. Four collagen gel concentrations (0.5, 1.0, 1.5, 2.0 mg/mL) with cell concentration of 1.0×10⁶ cells/mL and the cell-gel mixture volume of 2 mL were evaluated. Cellular distribution was assessed by observing labeled nuclei and actin with laser confocal microscope at 0.5, 1, and 2 days, respectively. BMSC-seeded gels were evaluated for their mechanical properties. It was observed that the rate of contraction decreased with higher initial collagen concentration. See FIG. 6. Lower concentrations of gelatin (0.5%) showed superior results in mechanical properties (FIG. 7). Images of cellular distribution at different time points showed that the gel contraction pattern with different collagen concentrations revealed the same contraction pattern (FIG. 8). The effects of cell density (0.1, 0.25, 0.5, and 1.0×10⁶ cells/mL) on the gel contraction rate and contracted gel ring mechanical properties were evaluated. It was observed that high cell density (over 0.6×10⁶ cells/mL) correlated with faster gel contraction and superior mechanical properties (FIGS. 9 and 10).

A series of experiments were performed to investigate whether an engineered gel patch seeded with BMSC improved healing when implanted at a surgical tendon repair site. Forty canine flexor digitorum profundus (FDP) tendons were harvested and divided into four groups: tendons with gel patch alone without BMSC and cultured for 2 weeks; tendons with gel patch alone without BMSC and cultured for 4 weeks; tendons with gel patch seeded with BMSC and cultured for two weeks; and tendons with gel patch seeded with BMSC and cultured for four weeks. A total of 8 tendons in each group were tested with ultimate failure strength (UFS). Two tendons from each group were used for cell viability assessment.

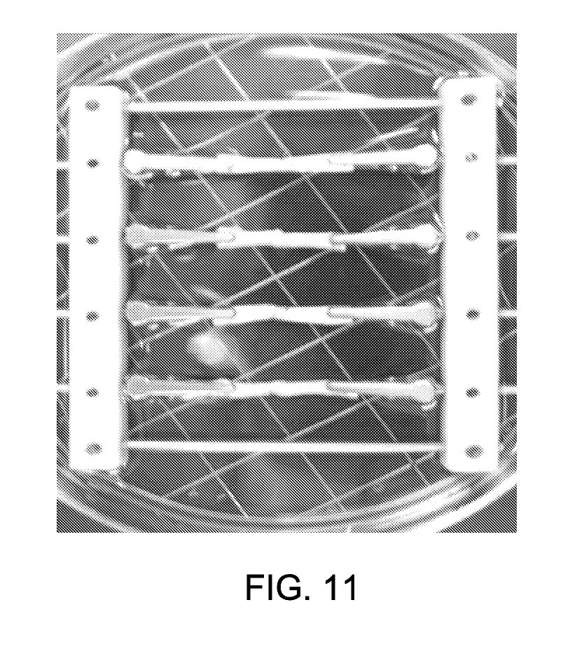

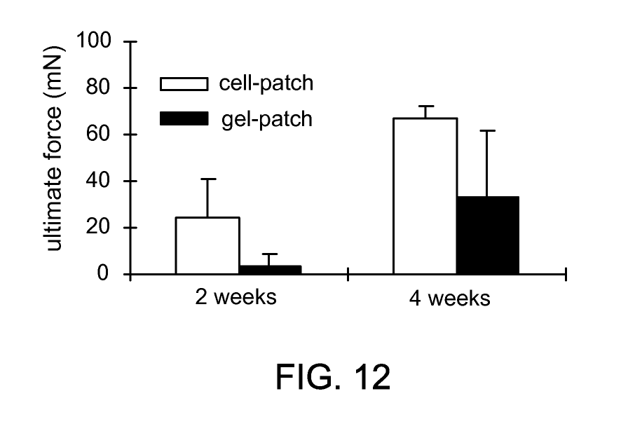

Isolated canine BMSC, at an initial concentration of 1×10⁶ cells, were seeded into 0.5 mg/mL of collagen. In order to assess the cell viability and distinguish the BMSC from the tenocytes existing in the native tendon, the BMSC were labeled with PKH26 red fluorescent cell linker before seeding to the gel patch. The cell-seeded gel was cultured for one day and then implanted between the cut tendon ends at the time of surgical repair. The repaired tendons were mounted on a square frame with 4 pairs of clamps to maintain tendon in a straight position during tissue culture (FIG. 11). After culturing for two- and four-weeks post-implantation, the repaired tendon was connected via a single suture at each end to a custom-designed micro-tester for mechanical evaluation. Before the testing, the repair sutures were cut carefully, without disrupting the repair site. In this way, healing strength, rather than suture strength, could be assessed. Following tissue culture, the tendon samples were examined by confocal microscopy.

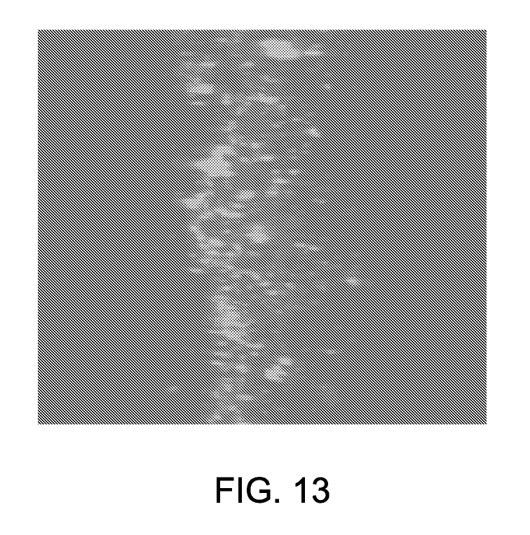

It was observed that the ultimate failure strength of the tendons repaired with the cell-seeded patch was significantly higher than that of the gel patch alone (p<0.05) at two- and four-weeks post-implantation. The strength of the repaired tendons at four weeks was significantly higher than at two weeks post-implantation in both gel alone and cell-seeded groups (p<0.05) (FIG. 12). BMSC labeled with red fluorescent on the cell membrane showed that cells were viable after two weeks in tissue culture (FIG. 13). These results indicated that a gel patch seeded with bone marrow stromal cells could accelerate tendon healing in an ex vivo tissue culture model.

### Example 4

### Effects of GDF-5 on Bone Marrow Stromal Cell Transplants

### A. Experimental Design

To investigate the effects of a BMSC seeded gel patch combined with GDF-5 on tendon healing using an in vitro tissue culture model, the following assays were performed. All tissues were obtained from mixed-breed dogs weighing between 20 and 30 kg. The animals were sacrificed for other, IACUC approved, studies. These studies involved tendon surgery on one forepaw. For this study, tissue from the hind paws was harvested.

Immediately after euthanasia, 8.0 mL of bone marrow was aspirated from each tibia using a 15 mL syringe containing 2.0 mL of heparin solution. Heparin was removed and the bone marrow cells from one dog were divided into three 100-mm dishes in 10 mL of standard medium, which consists of minimal essential medium (MEM) with Earle's salts (Gibco, Grand Island, N.Y.), 10% fetal calf serum and 1% antibiotics (Antibiotic-Antimycotic, Gibco). The bone marrow cells were incubated at 37° C. with 5% CO₂ and 95% air at 100% humidity. After 3 days, the medium containing floating cells was removed and new medium was added to the remaining adherent cells. These adherent cells were considered to be BMSCs. The medium was changed every 3 days. After the BMSCs formed colonies, they were treated with EDTA-trypsin to produce a cell suspension and centrifuged at 1500 rpm for 5 minutes to remove the EDTA-trypsin solution. The concentrated cell suspension was gathered in one tube and seeded in new dishes. Recombinant human GDF-5 (MBL, Woburn, Mass.) was added to the culture medium at a concentration of 100 ng/mL and culture continued for and additional 10 days.

Quantification of cell proliferation and viability was measured using Cell Proliferation Kit I (Roche, Basel, Switzerland). Briefly, BMSCs were seeded in micro-plates and cultured in medium supplemented with 100 ng/mL rhGDF-5 for 3 to 10 days. After the culture period, 10 μL of the MTT labeling regent was added to each well. The micro-plates were incubated at 37° C. in a 5% CO₂ humidified incubator for 4 hours. 100 μL of the solubilization solution was added into each well. Samples were incubated at 37° C. in a 5% CO₂ humidified incubator overnight. The absorbance was measured using Spectra Max Plus (Molecular Devises, Sunnyvale, Calif.). The wavelength was 570 nm.

Total RNA was extracted from culture cells using RNeasy Micro kit (Qiagen, Valencia, Calif.), according to the manufacturer's recommendations. The RNA concentration was determined using a NanoDrop (Thermo scientific, Waltham, Mass.). RNA was reverse transcribed into single-stranded cDNA with an anchored-oligo(dT) primer using Transcriptor First Strand cDNA Synthesis Kit (Roche). The reverse transcriptase was inactivated by heating to 85° C. for 5 minutes. The expression of tenomodulin (a marker of tenocyte differentiation), collagen type I and collagen type III was quantified with LightCycler 480 SYBR Green I Master kit (Roche) in a LightCycler 480 instrument (Roche). HPRT served as the reference gene. The PCR primers, designed from canine-specific cDNA sequences, are listed in Table 1. Five samples were measured in each group.

PureCol bovine dermal collagen (2.9 mg/ml, Inamed Corp., Fremont, Calif.) was prepared following the company's instructions. Briefly, 5.17 mL of sterile, chilled PureCol collagen was mixed with 3 mL of sterile 5× MEM, 0.35 mL of sterile 0.5M NaOH and 6.48 mL distilled H2O to adjust the pH to 7.4±0.2, making 15 ml temporary collagen/MEM solution on ice. The solution was then stored at 4-6° C. for no longer than 1 hour until use.

Confluent plates of BMSCs were washed with sterile PBS and then trypsinized. The cells were counted with a hemocytometer and centrifuged to remove the media and leave behind a cell pellet with a known number of cells. The amount of collagen and cell density was then adjusted to a final collagen concentration of 0.5 mg/mL and initial cell density 1.0×10⁶ cells/mL. A 2 mL aliquot of the cell-seeded collagen solution was added to a sterile 35 mm Petri dish. Evenly distributed over the surface, this would produce a 1 mm thick layer of solution. After incubating at 37° C. in a 5% CO₂ humidified incubator for one day for gelation, the BMSC-seeded collagen was cut to a similar cross-sectional shape as the tendon ends (roughly 2×4 mm) and used immediately. For the patch control group, collagen gel was prepared similarly, without the addition of BMSC in the final stages. For the growth factor stimulation group, the BMSC gel was mixed with rhGDF-5 at the concentration of 100 ng/mL.

The 2nd-5th digit FDP tendons were harvested under sterile conditions after animal sacrifice. For orientation purposes, the distal edge of the A2 pulley was marked prior to excision. Each tendon was transected 6 mm distal to the previously marked level and shortened by cutting to a standardized length of 30 mm, with the repair site located centrally. This section of the FDP tendon consists of two collagen bundles. The tendons were randomly assigned into four groups: 1) repaired tendon without gel patch; 2) repaired tendon with cell-seeded gel patch; 3) repaired tendon with GDF5 added gel patch without cells; and 4) repaired tendon with GDF5 treated cell-seeded gel patch. The gel patch was placed between the lacerated tendon ends. Then the tendon ends were sutured with two simple sutures of 6-0 Prolene (Ethicon, Somerville, N.J.).

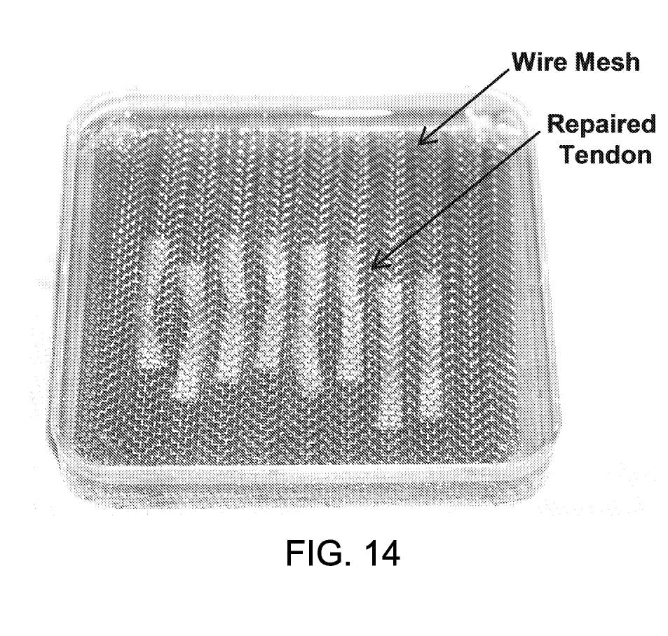

The repaired tendons were mounted on a wire mesh designed to maintain the tendons in a straight position (FIG. 14). The mesh was then placed into a 100 mm Petri dish with MEM with Earle's salts (Gibco), 10% fetal calf serum and 1% antibiotics (Antibiotic-Antimycotic, Gibco), and incubated at 37° C. in a 5% CO₂ humidified incubator for 2 or 4 weeks. Culture medium was changed every 3 days.

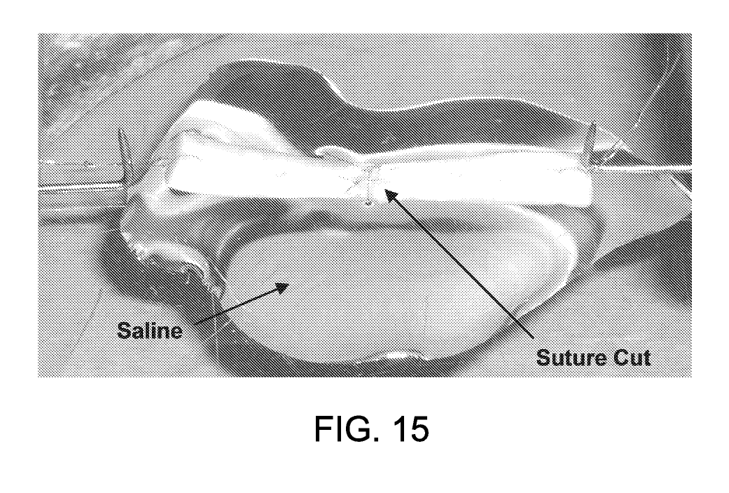

After culture, tendons (n=8) were removed from the culture dish and test specimens 30 mm in length were prepared, with the repair site in the middle. A single loop suture was placed at each end of the test specimen to connect the tendon to a custom-designed micro-tester for mechanical evaluation. The testing apparatus included a load transducer (Techniques Inc., Temecula, Calif.) which connected to the one of tendon loop and a motor and potentiometer (Parker Hannifin Corp., Rohnert Park, Calif.) which connected to the other loop. The loop at each tendon end was 5 mm long, so that the whole testing specimen including the repaired tendon and suture loops was 40 mm long. Before testing, the tendon repair sutures were cut, without disrupting the repair site, in order to assess the strength of the healing tissue rather than the suture strength (FIG. 15). For mechanical testing, the tendon was placed on a flat glass platform moistened with saline. The specimen was then distracted at a rate of 0.1 mm/second until the repair site was totally separated. The displacement and maximum strength measured by the transducer were recorded for data analysis.

From each test group, four tendon segments, including the repair site, were collected and fixed in 10% neutral buffered formalin. The tendon samples were then dehydrated and embedded in paraffin. Sections of 5 μm were cut in the sagittal plane using a Leica microtome (Leica Microsystems, Wetzlar, Germany). The sections were stained with hematoxylin and eosin (H&E) and then mounted on glass slides. The morphology and cellularity was evaluated with light microscopy. The results of MTT assay and RT-PCR were analyzed by unpaired t-test. The results of ultimate force and stiffness were analyzed by two-way ANOVA. A P-value of 0.05 or less was chosen to indicate significant difference between groups.

### B. Results

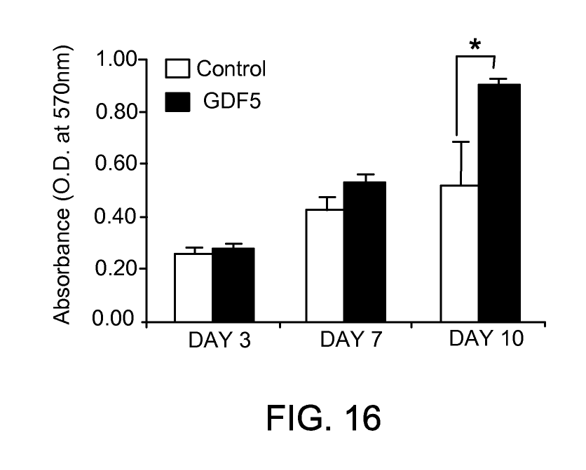

The proliferation of BMSCs with GDF-5 stimulation was significantly increased at day 10 of cell culture compared to the BMSCs without GDF5 stimulation (FIG. 16).

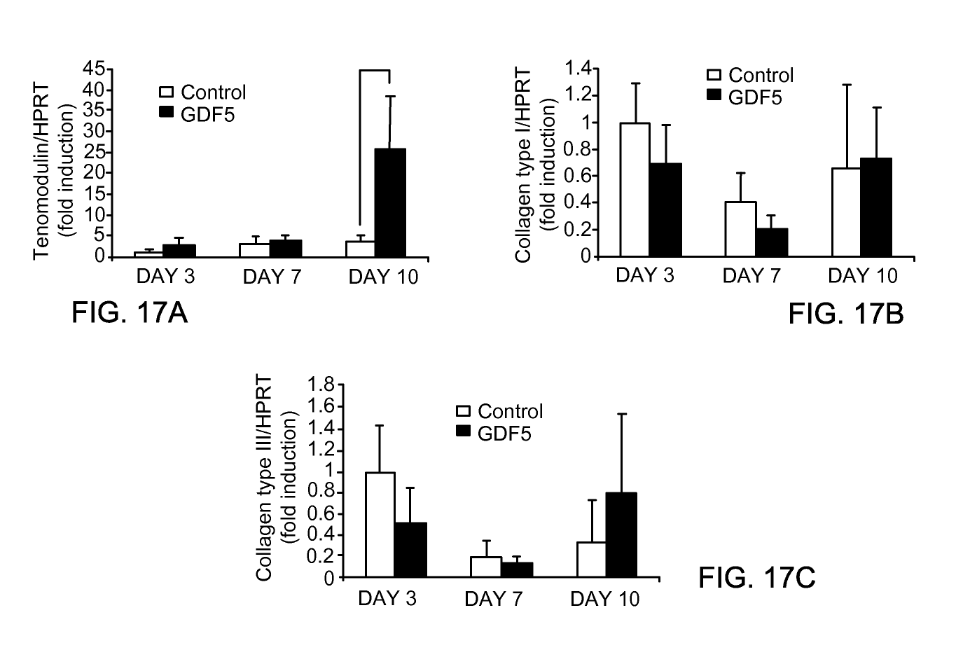

The expression of tenomodulin mRNA was increased in the cells treated with GDF-5 compared to the untreated cells at day 10. However no significant difference was found in collagen type I, or collagen type III mRNA expression in the cells treated with or without GDF-5 (FIG. 17).

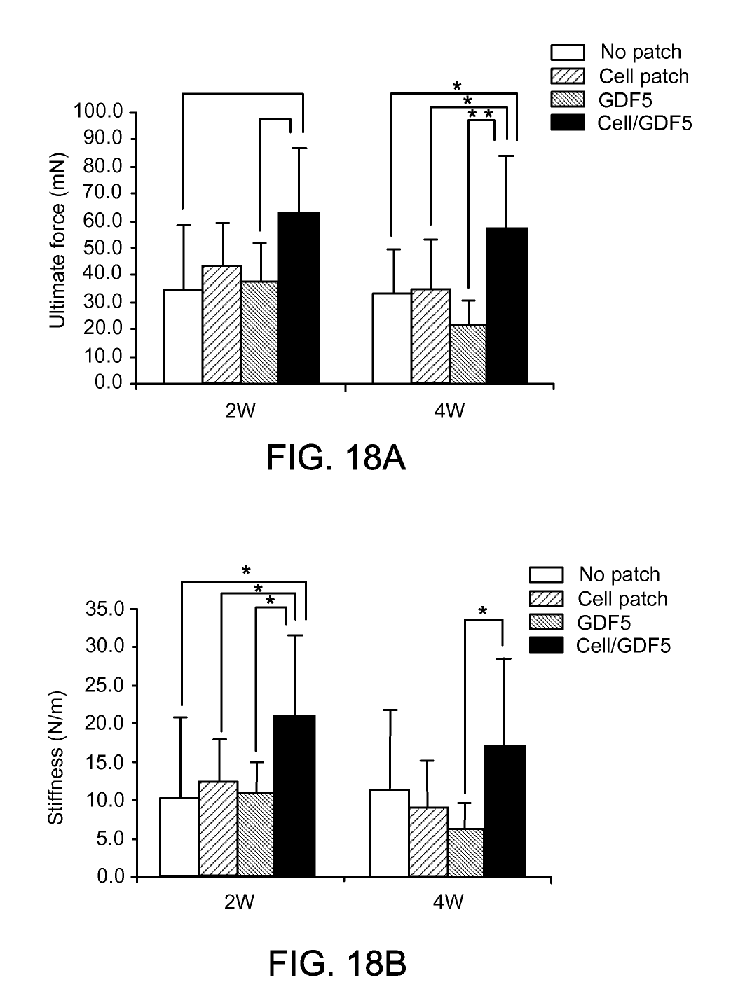

The maximum healing strength at two weeks was 34.3 (±23.9), 43.3 (±15.8), 37.4(±14.7), and 62.8 (±24.2) mN for repaired tendons without patch, with cell-seeded patch, with GDF-5 treated patch without cells, and with GDF-5 treated cell-seeded patch respectively. The maximum healing strength at four weeks was 32.9(±16.5), 34.1(±19.0), 21.3(±9.1), and 56.4 (±27.4) mN for repaired tendon without patch, with cell-seeded patch, with GDF-5 treated patch without cells and with GDF-5 treated cell-seeded patch respectively. The maximum healing strength with the GDF-5 treated BMSC-seeded patch was significantly higher than it was in tendons without a patch or with the patch with GDF-5 alone at 2 weeks (p<0.05). After 4 weeks in tissue culture, the maximum healing strength with the GDF-5 treated BMSC-seeded patch was significantly higher than it was for all other groups (p<0.05). There was no significant difference when comparing the strength of healing at 2 weeks and 4 weeks by repair type (FIG. 18).

The stiffness generally followed a similar pattern, i.e. the stiffness of the healing tendons treated with the GDF-5 treated BMSC-seeded patch was increased compared to other three groups. The stiffness of the healing tendons with the GDF-5 treated BMSC-seeded patch was significantly higher than other three groups at 2 weeks, but only significantly higher than the patch with GDF-5 alone at 4 weeks (p<0.05). There was no significant difference among the other three groups at either 2 or 4 weeks. No significant difference was detected between the 2 week and 4 week stiffness results in any group (FIG. 18).

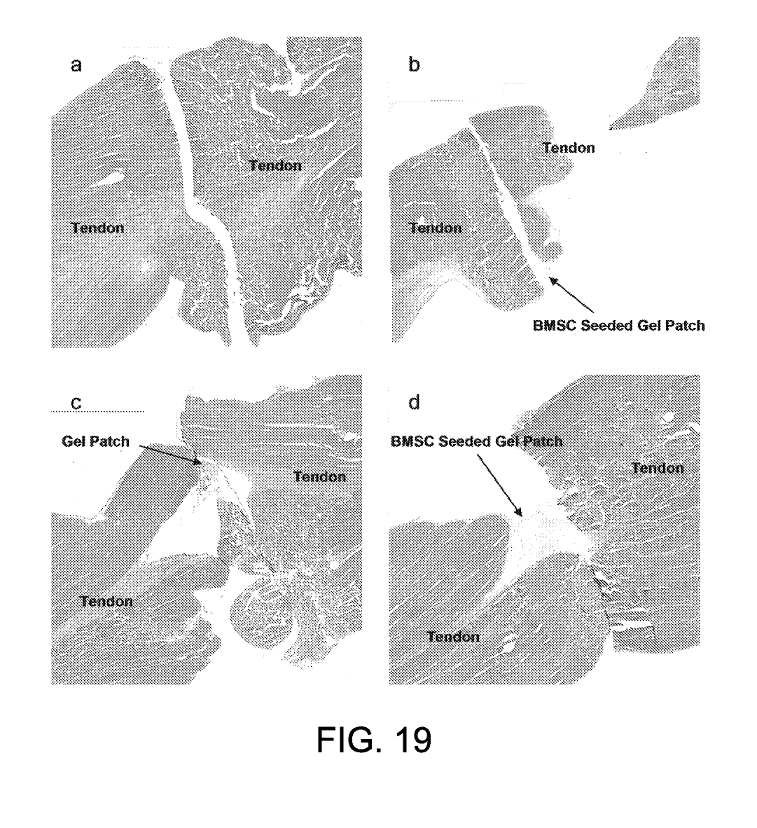

Qualitative observation by microscopy revealed that viable BMSCs were present between the cut tendon ends in GDF-5 treated cell-seeded gel patch group after four weeks in tissue culture. Partial healing was also found in the tendons repaired with a GDF-5 treated BMSC-seeded patch (FIG. 19

In sum, biomechanical testing showed that the maximal strength of healing tendons with a GDF-5 treated BMSC-seeded patch was significantly higher than in tendons without a patch. Histology also suggested better early healing with the GDF-5 treated BMSC-seeded patch. Thus, strength can indeed be improved with use of a BMSC patch and GDF-5. These results also support the potential of GDF-5 to accelerate tendon healing.

### Example 5

### Effects of Platelet-Rich Plasma on BMSC Transplants

In this study, the effect of platelet-rich plasma (PRP) and bone marrow-derived stromal cell (BMSC)-seeded interposition was investigated in an in vitro canine tendon repair model. Bone marrow, peripheral blood, and tendons were harvested from mixed breed dogs. BMSC were cultured and passaged from adherent cells of bone marrow suspension. PRP was purified from peripheral blood using a commercial kit. A total of 196 flexor digitorum profundus (FDP) tendons from the 2nd to 5th 15 digits of both forepaws and hind paws were immediately harvested from 13 dogs after sacrifice for other, IACUC approved, studies. The FDP tendons were then immediately immersed into cell culture medium to maintain tissue viability. The tendons were randomly assigned to one of four treatment groups and two time points, for a total of eight study groups with 24 tendons in each group (Table 1). Tendons repaired with a simple suture were used as a control group. In treatment groups, a collagen gel patch was interposed at the tendon repair site prior to suture. There were three treatment groups according to the type of collagen patch: a patch with PRP, a patch with BMSC, and a patch with PRP and BMSC. The repaired tendons were evaluated by biomechanical testing and by histological survey after 2 and 4 weeks in tissue culture. To evaluate viability, cells were labeled with PKH26 and surveyed under confocal microscopy after culture.

TABLE 1
Experimental Design

| | Culture 2 weeks | | Culture 4 weeks | |
|---|---|---|---|---|
| Groups | MT | HIS/CV | MT | HIS/CV |
| No patch | 20 | 4 | 20 | 4 |
| PRP alone patch | 20 | 4 | 20 | 4 |
| BMSC in collagen patch | 20 | 4 | 20 | 4 |
| BMSC in PRP patch | 20 | 4 | 20 | 4 |

* MT—mechanical testing; HIS—histological analysis; CV—cell viability analysis.

To prepare PRP, whole blood (55 mL) was withdrawn into a sterile syringe containing citric acid-citrate dextrose anticoagulant (ACD-A) at ratio of 10:1. The blood was then processed within 1 hour after harvest. PRP preparation from blood was carried out using the GPS III System (Biomet Biologic, Warsaw, Ind.), according to the manufacturer's directions. A solution of 1000 units of bovine thrombin (BioPharm, Alpine, Utah) per milliliter of 10% calcium chloride (Sigma, St. Louis, Mo.) was used to activate the PRP (see Pietrzak and Eppley, J Craniofac Surg (2005) 16(6):1043-1054), at a ratio of 6 mL of PRP to 1 mL of the thrombin/calcium chloride mix. This mixture was then left at room temperature for one hour to lyse the platelets and release the growth factors. The solutions were centrifuged for 5 minutes at 1500 rpm and the supernatant was used in the next step. Platelets within both whole blood and the PRP were counted for comparison according to the method of Brecher and Cronkite (J Appl Physiol (1950) 3(6):365-377). The mean platelet count in the PRP was 243×103/μl (range 198-324×10312/μl; SD 49×103/μl) and 1316×103/μl (range 919-1594×103/μl; SD 263×10313/μl) (p=0.0006). Platelet counts were 5.41-fold greater in the PRP compared to whole blood (range 4.15-6.75-fold 15 increase; SD 1.07).

BMSC were harvested and suspended as described above. BMSC in passage 3 were washed twice with sterile PBS and trypsinized. The cells were counted with a hemocytometer and centrifuged to remove the media and leave behind a cell pellet with a known number of cells. The amounts of collagen and cell density were adjusted to a final collagen concentration of 0.5 mg/mL and initial cell density 1.0×10⁶ cells/mL. A 2 mL aliquot of the cell-seeded collagen solution was added to a sterile 35 mm Petri dish. After incubating at 37° C. in a 5% CO₂ humidified incubator for one day for gelation, the BMSC-seeded patch was cut to a similar cross-sectional shape as the tendon ends (roughly 2×4 mm), and used immediately.

To prepare a BMSC-seeded PRP patch, BMSCs in Passage 3 were washed twice with sterile PBS and trypsinized. The cells were counted with a hemocytometer and centrifuged to remove the media and leave behind a cell pellet with a known number of cells. The amount of collagen and cell density were adjusted to a final collagen concentration of 0.5 mg/mL and initial cell density 1.0×10⁶ 5 cells/mL using 1 mL of the PRP supernatant and 1 ml of the collagen solution described above. A 2 mL aliquot of the BMSC-seeded PRP collagen solution was added to a sterile 35 mm Petri dish. After incubating at 37° C. in a 5% CO₂ humidified incubator for one day for gelation, the gel was cut and used immediately. For the PRP patch group, the PRP patch was prepared similarly, but without the addition of BMSC.

Each tendon was transected 6 mm distal to the distal edge of A2 pulley and shortened by cutting to a standardized length of 30 mm, with the repair site located centrally at the zone II D level. The gel was placed between the lacerated tendon ends. Then the tendon ends were apposed with two simple loop sutures of 6-0 Prolene (Ethicon, Somerville N.J.). The repaired tendons were mounted on a wire mesh designed to maintain the tendons in a straight position. The mesh was then placed into a 100 mm Petri dish with 50 ml of minimal essential medium (MEM), Earle's salts (GIBCO, Grand Island, N.Y.), 10% fetal calf serum, and 1% antibiotics (Antibiotic-Antimycotic, GIBCO, Grand Island, N.Y.), and incubated at 37° C. in a 5% CO₂ humidified atmosphere. Tendons were cultured for 2 or 4 weeks. Culture medium was changed every 3 days.

After culture, tendons were removed from the culture dish. A single loop suture was placed at each end of the test specimen to connect the tendon to a custom-designed micro-tester for mechanical evaluation. The testing apparatus included a load transducer (Techniques Inc., Temecula, Calif.) which connected to the one of the tendon loops, and a motor and potentiometer (Parker Hannifin Corp., Rohnert Park, Calif.) which were connected to the other loop. Before testing, the tendon apposition sutures were cut, without disrupting the repair site, in order to assess the strength of the healing tissue rather than the suture strength. For mechanical testing, the tendon was placed on a flat plastic platform moistened with saline. The specimen was then distracted at a rate of 0.1 mm/second until the apposition site was totally separated. The displacement and maximum strength measured by the transducer were recorded for data analysis. Cell viability analysis was performed as described above.

From each test group, four tendon segments, including the repair site, were collected and fixed in 10% neutral buffered formalin for 24 hours. The tendon samples were soaked in 10% to 20% of sucrose/0.1M PBS solution gradually. Sections of 6 μm were cut in the sagittal plane using a cryostat (Leica, Bannockburn, Ill.). The sections were mounted on glass slides and stained with hematoxylin and eosin (H&E). The morphology and cellularity were evaluated with light microscopy.

Analysis using a 2-factor ANOVA with repeated measures showed that the repaired method had a significant effect on both maximum strength and stiffness of the healing tendons with the repair sutures removed. The effect of time was not significant in either maximum strength or stiffness. Since the interaction between repair method and time was not significant, the comparison between each patch method was tested using the Tukey-Kramer post-hoc test.

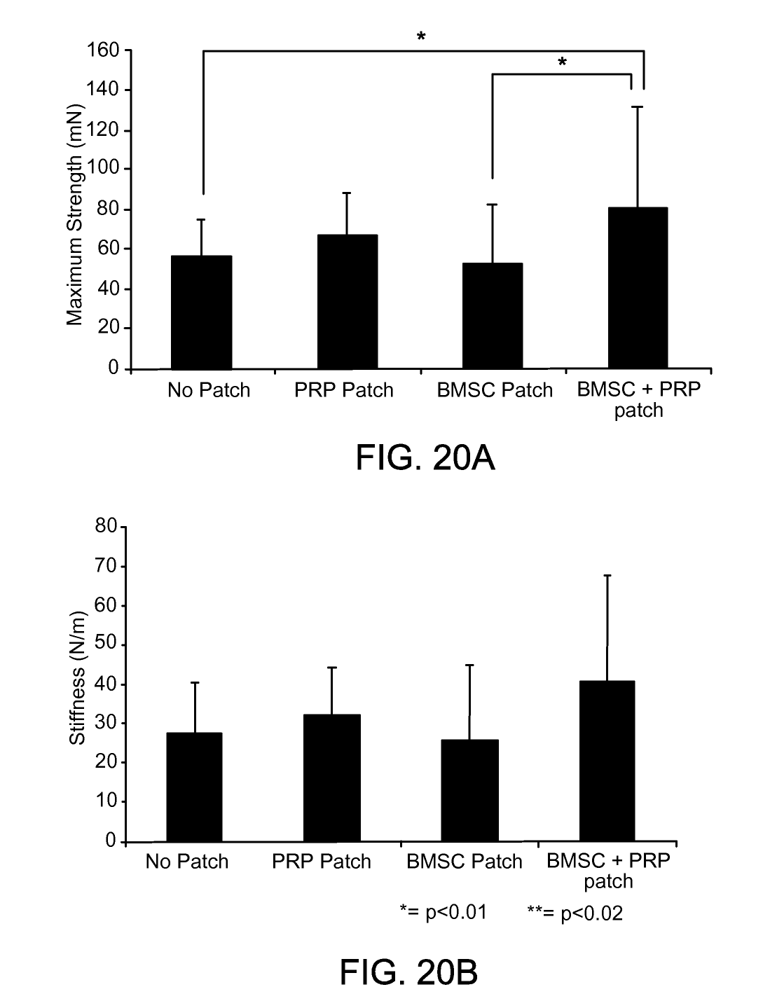

The maximum breaking strength of the healing tendons was 55.6 mN (SD 19.1), 67.0 mN (SD 21.3), 52.4 mN (SD 30.3), and 80.9 mN (SD 50.3) for the tendons without a patch, with a PRP patch, with a BMSC-seeded patch, and with a BMSC-seeded PRP patch, respectively (FIG. 20A). The maximum strength of the healing tendons with the BMSC-seeded PRP patch was significantly higher than the healing tendons without a patch (p=0.0077) or with a cell seeded patch (p=0.0025). The maximum strength of the healing tendons with a BMSC-seeded PRP patch was higher than the healing tendons with a PRP patch, but the difference was not statistically significant (p=0.16). The stiffness of the healing tendons followed a similar trend. The stiffness of the healing tendons was 27.5 N/m (SD 12.8), 31.7 N/m (SD 12.1), 25.7 N/m (SD 19.2), and 40.6 N/m (SD 27.1) for healing tendon without a patch, with a PRP patch, with a BMSC-seeded patch, and with a BMSC-seeded PRP patch, respectively (FIG. 20B). The stiffness of the healing tendons with a BMSC-seeded PRP patch was significantly higher than the healing tendons without a patch (p=0.018) or with a cell-seeded patch (p=0.0062).

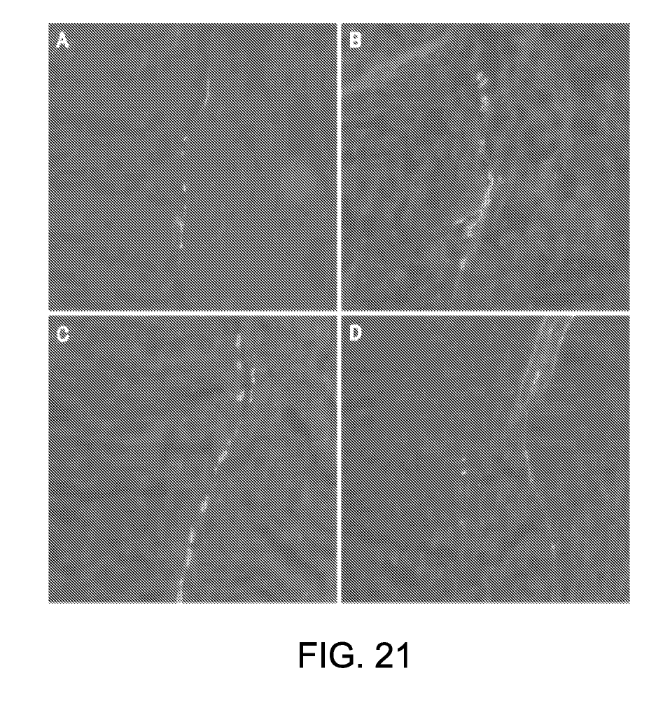

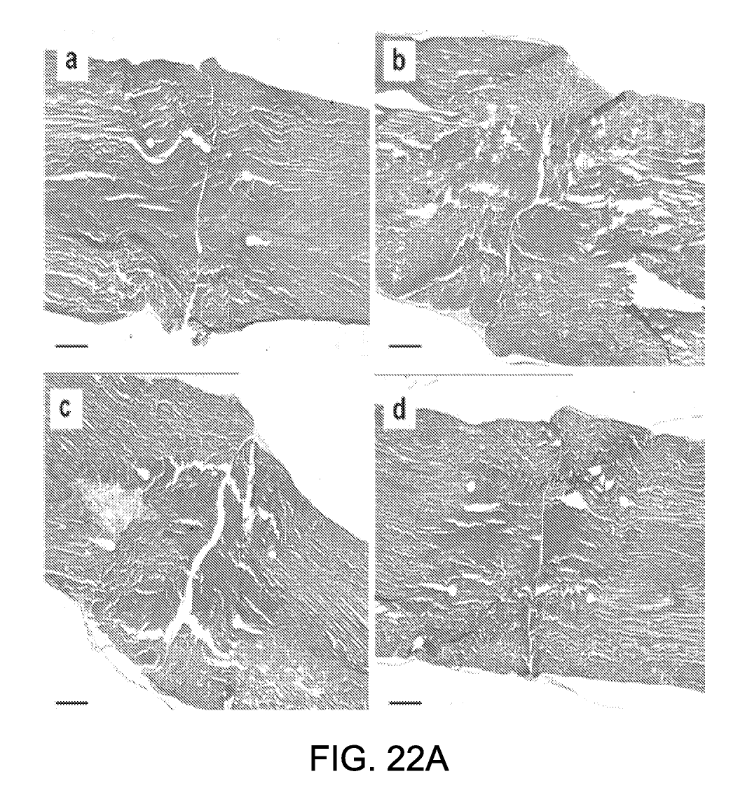

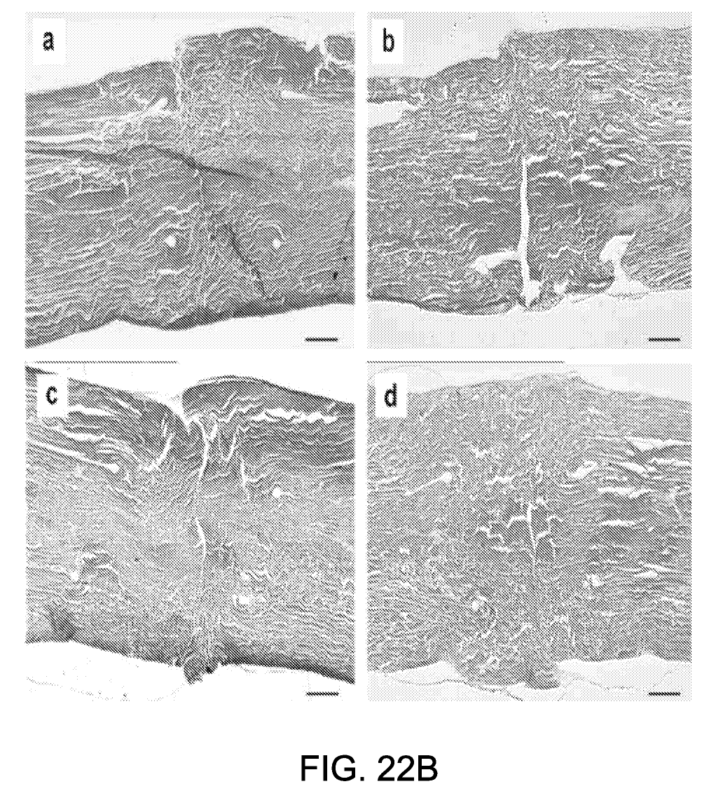

Qualitative observation by confocal microscopy revealed that labeled viable BMSCs were present among the repair site in both the BMSC-seeded patch and BMSC-seeded PRP patch group after 2 and 4 weeks of tissue culture (FIG. 21). No obvious difference was observed among those two groups. Under light microscopy, the healing site was bridged with an epitenon cell layer in all groups cultured for 2 weeks, but there was no evidence of healing within the tendon substance (FIG. 22A).

In the 4 week samples, epitenon cells bridged the healing site in all groups as in the 2 weeks samples, and in addition every sample showed partial healing between the tendon ends (FIG. 22B).

In sum, it was observed that the maximal strength of healing tendons was significantly higher with a BMSC patch with PRP than with no patch or a BMSC only patch. The BMSC patch with PRP improved maximal strength and stiffness of the healing tissue between the tendon ends in vitro. This result supports the potential of PRP to augment the tendon healing with a BMSC patch. The force measured between the healing tendon ends was in the order of mN. This is much lower than the force measured in a sutured tendon, usually a value several orders of magnitude larger, as is typically done in vivo studies of tendon healing, or in cadaver studies of different suture designs. The difference from such studies is that the current study is an attempt to gain insights regarding early tendon-tendon healing, in an attempt to hasten the time when the strength of the healing tendon exceeds the strength of the suture material used to support the healing tendon. In order to do this, the suture effect must be eliminated. What has been observed is that these initial healing strengths are very low, at least in the present tissue culture model. Of course it is also known that over 6-12 weeks in vivo the strength of the healing tissue between the tendon ends, even though it starts out at the mN level, grows by four orders of magnitude, eventually exceeding that of the tendon suture. Hopefully these low initial strengths can be increased, both with new stimuli and over time, especially in vivo, to hasten the moment when the tendon strength exceeds the suture strength, and the patient with a tendon injury can safely resume full activity. As a start, the present data have shown that the strength can indeed be improved with use of a BMSC patch with PRP.

### Example 6

### Effect of Substance P on BMSC Transplants

### A. Experimental Design

To investigate the effects of a BMSC seeded gel patch combined with Substance P on tendon healing using an in vitro tissue culture model, the following assays were performed. All tissues were obtained from mixed-breed dogs weighing between 20 and 30 kg.

Mixed-breed dogs being euthanized for other IACUC approved studies were used to harvest bone marrow from which bone marrow stromal cells (BMSCs) were cultured. The FDP tendons were harvested from dogs also being euthanized for other IACUC approved studies and these tendons were transected at similar levels, repaired with a single suture and incubated in tissue culture medium. Six repair groups was studied: (1) tendons ends opposed with two single loop sutures of 6/0 nylon (Ethicon, Somerville N.J.); (2) tendons repaired as in group 1 with the interposition of a collagen gel scaffold to the repair site (positive control group); (3) tendons repaired as in group 1 with the interposition of a BMSC-seeded collagen gel scaffold to the repair site; (4) tendons repaired as in group 1, with the interposition of a BMSC-seeded collagen gel scaffold to the repair site and addition of Substance P; (5) tendons repaired as in group 1, with the interposition of a BMSC-seeded collagen gel scaffold to the repair site and addition of GDF-5; and (6) tendons repaired as in group 1, with the interposition of a BMSC-seeded collagen gel scaffold to the repair site and addition of SP and GDF-5.

Immediately after euthanasia, 4.0 mL of bone marrow was aspirated from each tibia using a 20 mL syringe containing 1.0 mL of heparin solution. The same volume of PBS was added into the collected bone marrow, which will then be centrifuged at 1500 rpm for 5 minutes at room temperature. PBS and heparin was removed, and the bone marrow cells from one dog were divided into four 100-mm dishes in 10 mL of standard medium which consists of minimal essential medium (MEM) with Earle's salts (GIBCO, Grand Island, N.Y.), 10% fetal calf serum, and 5% antibiotics (Antibiotic-Antimycotic, GIBCO, Grand Island, N.Y.). The bone marrow cells were incubated at 37° C. with 5% CO₂ and 95% air at 100% humidity. After 5 days, the medium containing floating cells was removed and new medium was added to the remaining adherent cells. The medium was changed every other day. After the adherent cells reached confluence, BMSC were treated with EDTA-trypsin to produce a cell suspension, and then centrifuged at 1500 rpm for 5 minutes to remove the EDTA-trypsin solution. The concentrated cell suspension was collected in one tube and the concentration of cell suspension will then be adjusted to 5.0×10⁶ cells/mL by adding medium.

Vitrogen bovine dermal collagen (Cohesion Technologies, Palo Alto, Calif., U.S.A.) was prepared following the manufacturer's instructions. Briefly, 10 mL of sterile, chilled Vitrogen collagen was mixed with 3 mL of sterile 5× MEM, 1.05 mL of sterile 0.167M NaOH, and 0.95 mL distilled H₂O to adjust the pH to 7.4±0.2, making 15 mL temporary collagen/MEM solution on ice. The solution was then stored at 4-6° C. for no longer than 1 hour before use.

Confluent plates of BMSC were washed twice with sterile PBS and then trypsinized. The cells were counted with a hemocytometer and centrifuged to remove the media, forming a cell pellet with a known number of cells. The amount of collagen and cell density will then be adjusted to a final collagen concentration of 0.5 mg/mL, and initial cell density 1.0×10⁶ cells/mL. A 1 mL aliquot of the cell-seeded collagen solution was added to a sterile 35 mm Petri dish. Evenly distributed over the surface, this will produce a 1 mm thick layer of solution.

After incubating at 37° C. in a 5% CO₂ humidified incubator for one day for gelation, the BMSC-seeded collagen was cut to a similar cross-sectional shape as the tendon ends (roughly 2×4 mm), and used immediately. For the patch control group, collagen gel was prepared similarly, without the addition of BMSC in the final stages. For the growth factor stimulation group, the BMSC gel was prepared with substance P (1×10⁻⁹ M or 10⁻⁶M) (Sigma).

The laceration of the flexor digitorum profundus (FDP) tendon was made at the PIP joint level, where the FDP tendon is composed of two fibrous bundles The gel patch was placed between the lacerated tendon ends. Then the tendon ends was sutured with two single loop sutures of 6/0 nylon (Ethicon, Somerville N.J.). The repaired tendons were mounted on a square frame with 4 pairs of clamps designed to maintain the tendons in a straight position. The frame was then placed into a 100 mm Petri dish with minimal essential medium (MEM), Earle's salts (GIBCO, Grand Island, N.Y.), 10% fetal calf serum, and 5% antibiotics (Antibiotic-Antimycotic, GIBCO, Grand Island, N.Y.), and incubated at 37° C. in a 5% CO₂ humidified atmosphere for two or 4 weeks. Culture medium was changed every 72 hours.

After culture, tendons (n=8) was removed from the culture dish and test specimens 30 mm in length was prepared, with the repair site in the middle. A single loop suture was placed at each end of the test specimen to connect the tendon to a custom-designed micro-tester for mechanical evaluation. Before testing, the tendon repair sutures was cut, without disrupting the repair site, in order to assess the strength of the healing tissue rather than the suture strength.

In order to assess the cell viability and distinguish the BMSC from the tenocytes existing in the native tendon, for four patches in each group the BMSC was labeled with PKH26 red fluorescent cell linker (Sigma, St. Louis, OM) before seeding in the gel patch. The patch with the labeled BMSC was implanted between the cut tendon ends following the same procedure described above. After tissue culture for 2 or 4 weeks, the tendon samples was observed with confocal microscopy (LSM510 Zeiss, Germany) to assess the viability of the transplanted BMSC.

From each test group, four tendon segments, including the repair site, was collected and fixed in 10% neutral buffered formalin. The tendon samples will then be dehydrated and embedded in paraffin. Sections of 5 μm was cut in the sagittal plane using a Leica microtome. The sections was stained with hematoxylin and eosin (H&E), and then mounted on glass slides. The morphology and cellularity was evaluated with light microscopy.

Stress at maximum force and stiffness were analyzed with Kruskal-Wallis nonparametric analysis of variance (ANOVA) followed by Mann-Whitney unpaired tests for comparisons between groups. For intragroup comparison, the Friedman 2-way ANOVA test was used, followed by a pairwise comparison using Wilcoxon matched-pairs signed-rank test. The significance level was predetermined at an alpha of 0.05.

### B. Results

The results of maximum force at failure and of stiffness are shown in Table 2. Zone II indicates the flexor tendons are within the flexor sheath, and zone III is the flexor tendon between distal carpal tunnel to proximal flexor sheath (i.e., proximal portion of Zone II). Maximum force was significantly higher in the zone III tendon groups with added Substance P at a concentration of 10̂-9 at both 2 weeks and 4 weeks. It should also be noted that zone III tendons overall tend to produce higher maximum forces relative to zone II at failure regardless of experimental group. In all groups, force at failure was significantly higher at 4 weeks compared to 2 weeks. Tendons in zone II did not produce statistically significant results relative to the cell only control. Large differences are noted in the zone III 4 week time-points but only at a Substance-P concentration of 10⁻⁹M. It is also noteworthy that there did not appear to be a difference between Cell/SP and SP only in any group at any time-point.

In this study, SP was found to have a proliferative effect on fibroblasts in the healing of ruptured rat Achilles' tendons from week 1 to week 6. Interestingly, the study did not find a statistical difference of maximum force in zone II FDP tendons, but a statistically significant difference was observed in zone III at two- and four-weeks relative to control.

TABLE 2
Biomechanical Testing Results

| Zone II Group | Week 2 | Week 4 | Zone III Group | Week 2 | Week 4 |
|---|---|---|---|---|---|
| Cell Only | 12.33 ± 2.87 | 25.87 ± 2.85 | Cell Only | 11.5 ± 2.3 | 37.38 ± 8.10 |
| Cell/SP [10⁻⁶] | 13.14 ± 7.68 | 24.14 ± 2.90 | Cell/SP [10⁻⁶] | 16.00 ± 3.42 | 37.06 ± 14.07 |
| Cell/SP ([10⁻⁹] | 16.77 ± 7.24 | 25.17 ± 18.51 | Cell/SP ([10⁻⁹] | 28.00 ± 9.65* | 242.15 ± 75.85* |
| SP Only [10⁻⁶] | 10.60 ± 8.14 | 20.833 ± 3.38 | SP Only [10⁻⁶] | 15.68 ± 7.05 | 33.57 ± 7.57 |
| SP Only [10⁻⁹] | 10..79 ± 3.37 | 27.25 ± 4.53 | SP Only [10⁻⁹] | 36.00 ± 13.33* | 218.30 ± 61.51* |

Values are mean millinewtons (mN) ± standard deviation (SD)
*Significant difference at the 0.05 level as compared with the control group in the same week.

The results of stiffness analysis are presented in Table 3. Stiffness is a representation of the slope in a load-extension curve; and it corresponds to how well a tendon withstands force. Statistically significant differences in stiffness parallel the differences of the maximum force. That is, zone II tendons did not produce statistically significant results relative to the cell only control. Large differences are noted in the zone III 4 week time-points but only at a Substance-P concentration of 10⁻⁹M. It is also noteworthy that there does not seem to be a difference between Cell/SP and SP only in any group at any time-point. In sum, stiffness followed force in that the week zone III 10-9M SP treated groups at both two and four weeks showed significant differences.

TABLE 3
Stiffness of Repaired Tendons

| Zone II | Week 2 | Week 4 | Zone III | Week 2 | Week 4 |
|---|---|---|---|---|---|
| Cell Only | 2.90 ± 1.11 | 7.39 ± 3.36 | Cell Only | 2.30 ± 1.31 | 10.28 ± 2.52 |
| Cell/SP [10\hat{ }−6] | 3.59 ± 1.66 | 7.26 ± 3.22 | Cell/SP [10\hat{ }−6] | 3.16 ± 1.68 | 10.01 ± 2.56 |
| Cell/SP [10\hat{ }−9] | 4.00 ± 1.56 | 5.68 ± 3.99 | Cell/SP [10\hat{ }−9] | 7.60 ± 2.55* | 82.20 ± 35.81* |
| SP Only [10\hat{ }−6] | 2.42 ± 1.35 | 7.22 ± 4.49 | SP Only [10\hat{ }−6] | 4.65 ± 2.29 | 5.35 ± 3.91 |
| SP Only [10\hat{ }−9] | 2.1 ± 1.06 | 4.53 ± 1.36 | SP Only [10\hat{ }−9] | 8.92 ± 5.06* | 57.82 ± 25.53* |

Values are mean N/mm ± standard deviation (SD).
*Significant difference at the 0.05 level as compared with the control group in the same week.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a tissue matrix and an anti-adhesive coating, wherein said tissue matrix comprises stem cells and one or more structural polypeptides or one or more biocompatible polymers, and wherein said anti-adhesive coating is present on at least one surface of said tissue matrix.

2. The composition of claim 1, wherein said coating is present on at least one surface of said tissue matrix that does not contact a wound or sutured tissue when implanted in a mammal.

3. The composition of claim 2, wherein said wound or sutured tissue is tendon, ligament, abdominal, uterine, or muscle tissue.

4. The composition of claim 1, wherein said one or more structural proteins are selected from the group consisting of a collagen, a proteoglycan, and a cytokine, or any combination thereof.

5. The composition of claim 1, wherein said one or more structural polypeptides are selected from the group consisting of collagen, aggregan, versican, decorin, biglycan, fibromodulin, lumican, IL-1, IL-6, and TNF-α, or any combination thereof.

6. The composition of claim 1, wherein said tissue matrix is an acellular tissue scaffold.

7. The composition of claim 1, wherein said tissue matrix is a collagen matrix.

8. The composition of claim 7, wherein said collagen matrix is a matrix of bioengineered collagen fibers.

9. The composition of claim 1, wherein said biocompatible polymer is a natural or synthetic biodegradable polymer.

10. The composition of claim 1, wherein said anti-adhesive coating is selected from the group consisting of lubricin, hyaluronic acid, phospholipids, or any combination thereof.

11. The composition of claim 10, wherein said lubricin is native human lubricin.

12-13. (canceled)

14. The composition of claim 1, wherein said stem cells are autologous stem cells.

15. The composition of claim 1, wherein said stem cells are derived from muscle, skin, bone marrow, synovium, or adipose tissue.

16. The composition of claim 1, wherein said stem cells are mesenchymal stem cells.

17. (canceled)

18. The composition of claim 1, wherein said composition is an implantable patch.

19. The composition of claim 1, further comprising a growth factor selected from the group consisting of transforming growth factor (TGF-β1), platelet derived growth factor (PDGF), basic fibroblast growth factor (b-FGF), insulin like growth factor (IGF), epidermal growth factor (EGF), growth differentiation factor-5 (GDF-5), growth differentiation factor 6 (GDF-6), growth differentiation factor 7 (GDF-7), and vascular endothelial growth factor (VEGF), or any combination thereof.

20. The composition of claim 1, further comprising a neuropeptide.

21. (canceled)

22. The composition of claim 1, further comprising platelet-rich plasma.

23-40. (canceled)

41. A method for treating a wound or sutured tissue comprising contacting a tissue matrix to said wound or sutured tissue, wherein said tissue matrix comprises one or more stem cells and one or more structural polypeptides or one or more biocompatible polymers, and coating at least a portion of said tissue matrix and/or tissue adjacent to said wound or sutured tissue with an anti-adhesive.

42-61. (canceled)

62. A method for treating a wound or sutured tissue comprising contacting a composition to said wound or sutured tissue, wherein said composition comprises a tissue matrix comprising one or more stem cells, one or more structural polypeptides or one or more biocompatible polymers, and an anti-adhesive coating, and whereby said wound or sutured tissue is treated.

63-90. (canceled)
